(19) United States
(12) Patent Application Publication
(10) Pub. No.: US 2011/0245174 A1
(43) Pub. Date: Oct. 6, 2011

(54) GLP-1 FUSION POLYPEPTIDES

(76) Inventors: Peter Artymiuk, Sheffield (GB); Richard Ross, Sheffield (GB); Jon Sayers, Chesterfield (GB)

(21) Appl. No.: 13/060,050

(22) PCT Filed: Aug. 18, 2009
(86) PCT No.: PCT/GB2009/002006

§ 371 Date: Mar. 16, 2011

Related U.S. Application Data
(60) Provisional application No. 61/090,813, filed on Aug. 21, 2008.

(30) Foreign Application Priority Data
Aug. 21, 2008 (GB) 0815248.0
May 7, 2009 (GB) 0907794.2
Aug. 10, 2009 (GB) 0913901.5

(51) Int. Cl. A61K 38/26; C07K 19/00; C07H 21/04; A61P 3/10; A61P 9/00; A61P 3/00; A61P 3/06

(52) U.S. Cl. 514/11.7; 530/399; 530/324; 536/23.4

(57) ABSTRACT

We describe nucleic acid molecules that encode fusion polypeptides comprising GLP-1, or a receptor binding part thereof, linked directly or indirectly to a polypeptide that naturally binds GLP-1.

The invention relates to a fusion polypeptide comprising a GLP peptide or functional variant thereof; dimers comprising said fusion polypeptide; and methods to treat diseases that would benefit from administration of said fusion polypeptide.

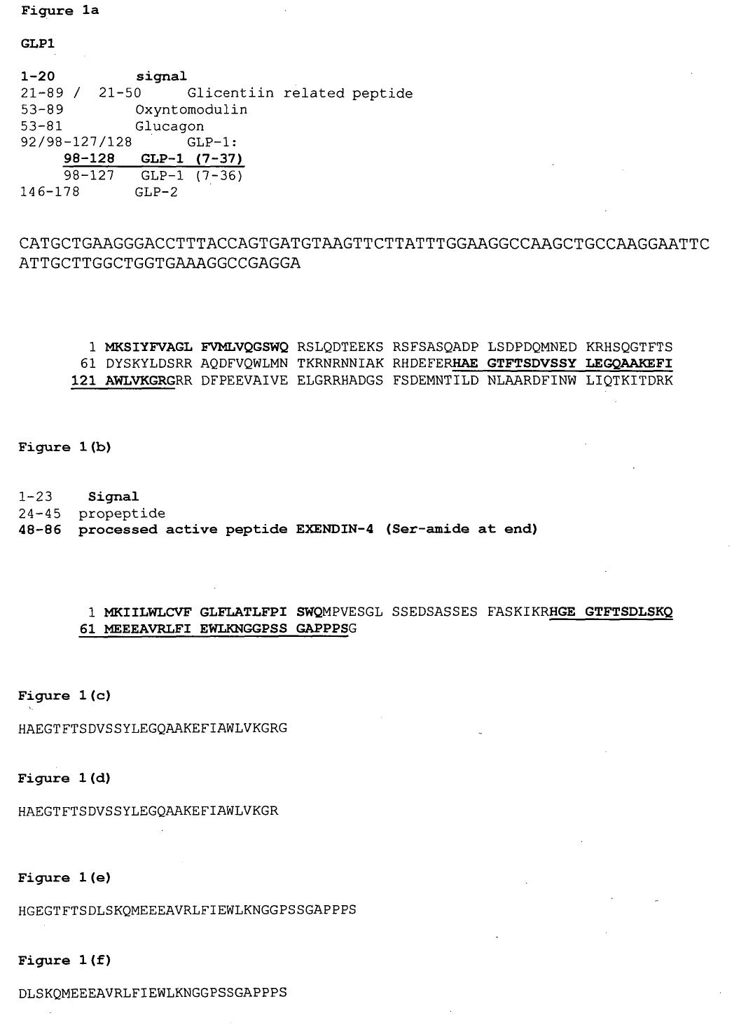

Glucagon-like peptide 1 [GLP-1] has various functions. For example GLP-1 stimulates the production of insulin by pancreatic β cells, enhances pancreatic β cell proliferation, inhibits pancreatic β cell apoptosis, lowers glucagon activity, slows gastric emptying and enhances insulin sensitivity. GLP-1 is derived from a larger polypeptide referred to as proglucagon which comprises glucagon [29 amino acids], GLP-1 [36 or 37 amino acid residues] and GLP-2 [34 amino acid residues], FIG. 1b. GLP-1 exists in two forms, a 37 amino acid peptide and a 36 amino acid peptide which is created by proteolytic cleavage by dipeptidyl peptidase IV [DPP4]. DPP4 binds an enzyme called adenosine deaminase [ADA] with high affinity. The significance of the association of DPP4 with ADA is unclear. However ADA is known to be associated with severe combined immunodefiency [SCID]. GLP-1 activates a GLP-1 receptor which is a G-coupled receptor [also known as a seven transmembrane receptor] expressed by pancreatic β cells and to a lesser extent by lungs, kidney, heart, gastro-intestinal tract and the brain.

There are a number of pathological conditions that result in hyperglycaemia; the most well known being diabetes mellitus. Diabetes mellitus can be of type I or type II. Type I diabetes is an autoimmune disease resulting in destruction of the pancreatic β cells which means the subject is unable to manufacture any insulin. Type II diabetes is a more complicated condition and can result from a number of associated ailments but commonly involves resistance to the metabolic actions of insulin. For example, type II diabetes is associated with age, obesity, a sedentary life style which results in insulin resistance. An associated condition is called Metabolic Syndrome which may predispose subjects to type II diabetes. The symptoms associated with this syndrome are high blood pressure, dyslipidemia, increased body fat deposition and cardiovascular disease. A further condition that results in insulin resistance is polycystic ovary syndrome which results in a failure to produce mature ova, androgen excess and hirsuitism. Hypoglycaemia [abnormally low levels of serum glucose] is also known and is typically the result of administration of an insulin overdose.

GLP-1 has been used as a therapeutic agent in the control of [symbol] problem associated with native GLP-1 is that because of its small mass it is very rapidly cleared from the circulation having a pharmacokinetic half life of 2-5 mins. This means that to achieve a therapeutic effect a relatively large dose of GLP-1 has to be administered. This has lead to the development of long acting forms of GLP-1 and the use of DPP4 inhibitors. The former approach involves the production of fusion proteins comprising GLP-1; the latter utilizes DPP4 inhibitors which suffer from a lack of specificity due to the fact that the inhibitor will inactivate DPP4s that modify other peptide hormones leading to undesirable side effects. There is therefore a continued desire to address the problem of rapid digestion and/or renal clearance of GLP-1 and related molecules.

As mentioned above prior art approaches to reduce rapid GLP-1 clearance involves the creation of GLP-1 fusion proteins. For example, WO2007/016764 describes a fusion protein comprising GLP-1 and an autoimmune suppressor to decrease an autoimmmune reaction in type I diabetes. EP1 724 284 describes the fusion of GLP-1 to either the Fc portion of an immunoglobulin or to albumin. Similarly, WO2005/00892 describes fusion proteins comprising GLP-1 analogues and the Fc portion of IgG4 and their use in the treatment of diabetes, obesity and irritable bowel syndrome. In US2007/0111940 are disclosed conjugates comprising GLP-1 and a peptide carrier that includes modified amino acids that improve stability of GLP-1. In U.S. Pat. No. 7, 716, 278 the fusion of GLP-1 to transferrin is used to reduce renal clearance and treat diabetes and related conditions. In WO2008/061355 an alternative to fusing GLP-1 to a carrier protein/peptide is described which is an implantable hydrogel device which releases GLP-1 and analogues of GLP-1 in a sustained fashion over a defined period.

This disclosure relates to alternative fusion polypeptides comprising a GLP-1 peptide or functional analogue thereof. In one embodiment GLP-1 is linked to an extracellular domain of a GLP-1 receptor. Alternative embodiments include the fusion of GLP-1 to inactivated DDP4 and optionally inactive ADA.

According to an aspect of the invention there is provided a nucleic acid molecule comprising a nucleic acid sequence that encodes a polypeptide that has the activity of GLP-1 wherein said polypeptide comprises GLP-1, or a receptor binding part thereof, linked directly or indirectly to a polypeptide that naturally binds GLP-1.

According to an aspect of the invention there is provided a fusion [symbol] the amino acid sequence of a GLP-1 peptide or functional analogue thereof, linked directly or indirectly to a polypeptide that naturally binds GLP-1.

In a preferred embodiment of the invention the polypeptide that naturally binds GLP-1 is the GLP-1 binding domain of the GLP-1 receptor.

In an alternative preferred embodiment of the invention the polypeptide that naturally binds GLP-1 is an enzymatically inactive GLP-1 dipeptidyl peptidase.

In a preferred embodiment of the invention said inactive GLP-1 dipeptidyl peptidase is modified by addition, deletion or substitution of at least one amino acid residue wherein said modification is to the active site of GLP-1 dipeptidyl peptidase.

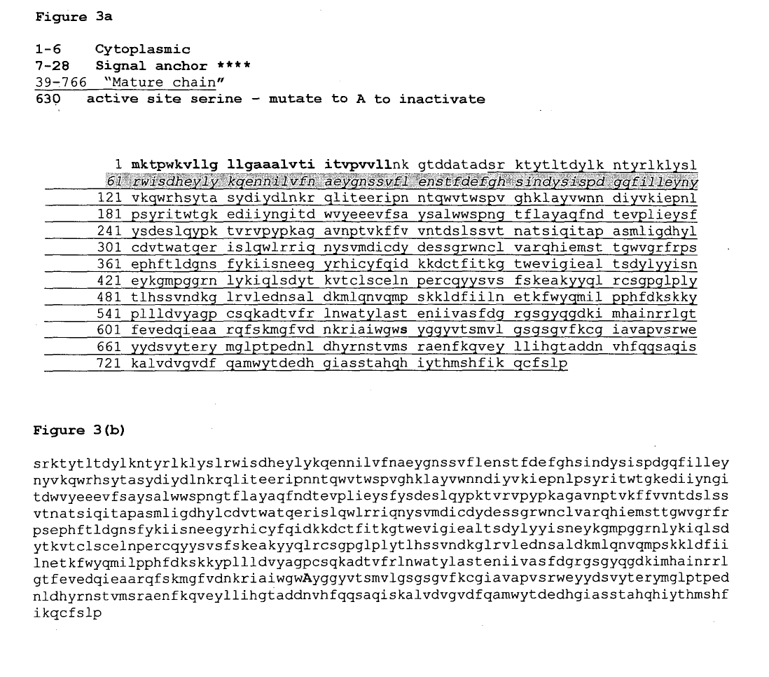

Preferably said modification is to amino acid residue 630 of the amino acid sequence represented in FIG. 3a.

In a preferred embodiment of the invention said fusion polypeptide comprises or consists of the amino acid sequence represented in FIG. 3b.

In a preferred embodiment of the invention said fusion polypeptide further comprises a polypeptide that naturally binds said GLP-1 dipeptidyl peptidase wherein said polypeptide is an enzymatically inactive adenosine deaminase.

In a preferred embodiment of the invention said inactive adenosine deaminase is modified by addition, deletion or substitution of at least one amino acid residue wherein said modification is to the active site of said inactive adenosine deaminase.

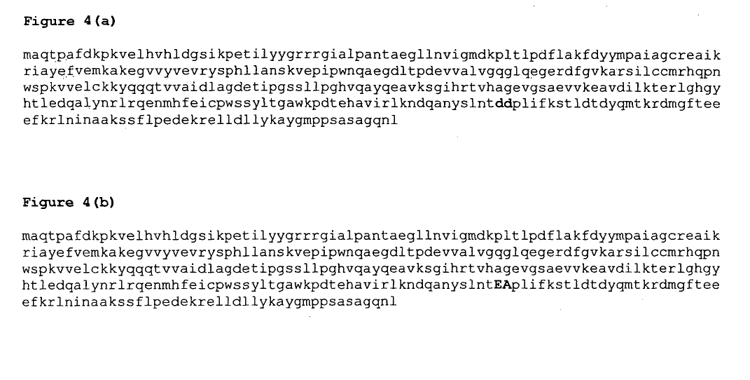

Preferably, said modification is to amino acid residues 295 and/or 296 of the amino acid sequence represented in FIG. 4a.

In a preferred embodiment of the invention said fusion polypeptide comprises or consists of the amino acid sequence represented in FIG. 4b.

In a preferred embodiment of the invention said fusion polypeptide comprises a GLP-1 peptide comprising or consisting of the amino acid sequence: HAEGTFTSDVSSYLEGQAAKEFIAWLVKGR, or a modified GLP-1 peptide wherein said modified peptide varies from said amino acid sequence by addition, deletion or substitution of at least one amino acid residue wherein said modified GLP-1 peptide retains or has enhanced GLP-1 activity when compared to an unmodified GLP-1 peptide.

In a preferred embodiment of the invention said GLP-1 peptide comprises the amino acid sequence:

HAEGTFTSDVSSYLEGQAAKEFIAWLVKGR;
or
HAEGTFTSDVSSYLEGQAAKEFIAWLVKGRG.

In a preferred embodiment of the invention said fusion polypeptide comprises an amino acid sequence:

HGEGTFTSDLSKQMEEEAVRLFIEWLKNGGPSSGAPPPS;
or
DLSKQMEEEAVRLFIEWLKNGGPSSGAPPPS.

In a preferred embodiment of the invention GLP-1 is linked to a polypeptide that naturally binds GLP-1 by a peptide linker.

In a preferred embodiment of the invention GLP-1 is linked to an inactive GLP-1 dipeptidyl peptidase GLP-1 by a peptide linker.

In a preferred embodiment of the invention GLP-1 is linked to an inactive adenosine deaminase by a peptide linker.

In a further preferred embodiment of the invention fusion inactive GLP-1 dipeptidyl peptidase is linked to an inactive adenosine deaminase by a peptide linker.

Preferably said peptide linker is a flexible peptide linker.

In a preferred embodiment of the invention said peptide linking molecule comprises at least one copy of the peptide Gly Gly Gly Gly Ser.

In a preferred embodiment of the invention said peptide linking molecule comprises 1, 2, 3, 4, 5, 6, 7, 8, 9, 10, 11 or 12 copies of the peptide Gly Gly Gly Gly Ser.

The polypeptide domains of the fusion polypeptide according to the invention typically are linked by peptide linkers as herein described, for example Gly₄Ser linkers. The number of copies of Gly₄Ser can vary. For example fusion of GLP-1 to DPP4 can vary between 0 and 10 copies, preferably 5-7 copies. The fusion of DDP4 to ADA domains can also vary from between 0 and 12 copies, preferably 7 or 8 copies. The fusion of GLP-1 to the ectodomain can vary between 0-8 copies, preferably 2-5 copies.

In an alternative preferred embodiment of the invention GLP-1 is linked to a polypeptide that naturally binds GLP-1 by a single peptidic bond.

In a preferred embodiment of the invention GLP-1 is linked to an inactive GLP-1 dipeptidyl peptidase GLP-1 by a single peptidic bond.

In a preferred embodiment of the invention GLP-1 is linked to an inactive adenosine deaminase by a single peptidic bond.

In a preferred embodiment of the invention inactive GLP-1 dipeptidyl peptidase is linked to an inactive adenosine deaminase by a single peptidic bond.

In an alternative preferred embodiment of the invention said peptide linker molecule comprises or consists of one copy of the glycosylation motif Asn-Xaa-Ser or Asn-Xaa-Thr where X is any amino acid except proline.

In a preferred embodiment of the invention said peptide linker molecule comprises at least 5 amino acid residues.

In a preferred embodiment of the invention said peptide linker comprises 5-50 amino acid residues.

In a further preferred embodiment of the invention said peptide linker consists of 5, 10, 15, 20, 25, 30, 35, 40, 45 or 50 amino acid residues.

In a preferred embodiment of the invention said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr.

In a preferred embodiment of the invention said peptide linker comprises at least one copy of an amino acid motif selected from the group consisting of:

Asn₁-Xaa₂-Ser₃ Xaa₄ Xaa₅ wherein Xaa₂ is any amino acid except proline;
Xaa₁ Asn₂-Xaa₃-Ser₄ Xaa₅ wherein Xaa₃ is any amino acid except proline;
Xaa₁ Xaa₂ Asn₃-Xaa₄-Ser₅ wherein Xaa₄ is any amino acid except proline;
Asn₁-Xaa₂-Thr₃ Xaa₄ Xaa₅wherein Xaa₂ is any amino acid except proline;
Xaa₁ Asn₂-Xaa₃-Thr₄ Xaa₅ wherein Xaa₃ is any amino acid except proline; and
Xaa₁ Xaa₂ Asn₃-Xaa₄-Thr₅wherein Xaa₄ is any amino acid except proline.

Preferably said peptide linker comprises at least one copy of a motif selected from the group consisting of:

Asn₁-Xaa₂-Ser₃ Gly₄ Ser₅ wherein Xaa₂ is any amino acid except proline;
Gly₁ Asn₂-Xaa₃-Ser₄ Ser₅wherein Xaa₃ is any amino acid except proline;
Gly₁ Gly₂ Asn₃-Xaa₄-Ser₅ wherein Xaa₄ is any amino acid except proline;
Asn₁-Xaa₂-Thr₃ Gly₄ Ser₅ wherein Xaa₂ is any amino acid except proline;
Gly₁ Asn₂-Xaa₃-Thr₄ Ser₅ wherein Xaa₃ is any amino acid except proline; and
Gly₁ Gly₂ Asn₃-Xaa₄-Thr₅wherein Xaa₄ is any amino acid except proline.

In an alternative preferred embodiment of the invention said peptide linker comprises at least one copy of a motif selected from the group consisting of:

Asn₁-Xaa₂-Ser₃ Ser₄ Gly₅ wherein Xaa₂ is any amino acid except proline;
Ser₁ Asn₂-Xaa₃-Ser₄ Gly₅wherein Xaa₃ is any amino acid except proline;
Ser₁ Ser₂ Asn₃-Xaa₄-Ser₅ wherein Xaa₄ is any amino acid except proline;
Asn₁-Xaa₂-Thr₃ Ser₄ Gly₅wherein Xaa₂ is any amino acid except proline;
Ser₁ Asn₂-Xaa₃-Thr₄ Gly₅wherein Xaa₃ is any amino acid except proline; and
Ser₁ Ser₂ Asn₃-Xaa₄-Thr₅wherein Xaa₄ is any amino acid except proline.

In a preferred embodiment of the invention said peptide linker molecule comprises at least one copy of the motif (Xaa₁, Xaa₂ Xaa₃ Xaa₄ Xaa₅) wherein said motif comprises the glycosylation motif Asn-Xaa-Ser or Asn-Xaa-Thr and at least one copy of the motif (Gly Gly Gly Gly Ser) wherein said peptide linker is 5-50 amino acids.

In a preferred embodiment of the invention said peptide linker comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and a copy of the motif (Ser Ser Ser Ser Gly) wherein said peptide linker is 5-50 amino acids.

In a preferred embodiment of the invention said fusion polypeptide linker is modified by the addition of at least one sugar selected from the group consisting of: mannose, galactose, N-acetyl glucosamine, N-acetyl neuraminic, acid N-glycolyl neuraminic acid, N-acetyl galactosamine, fucose, glucose, rhamnose, xylose, or a combinations of sugars, for example in an oligosacharide or scaffolded system.

Suitable carbohydrate moieties include monosaccharides, oligosaccharides and polysaccharides, and include any carbohydrate moiety that is present in naturally occurring glycoproteins or in biological systems. For example, optionally protected glycosyl or glycoside derivatives, for example optionally-protected glucosyl, glucoside, galactosyl or galactoside derivatives. Glycosyl and glycoside groups include both α and β groups. Suitable carbohydrate moieties include glucose, galactose, fucose, GlcNAc, GalNAc, sialic acid, and mannose, and oligosaccharides or polysaccharides comprising at least one glucose, galactose, fucose, GlcNAc, GalNAc, sialic acid, and/or mannose residue.

Any functional groups in the carbohydrate moiety may optionally be protected using protecting groups known in the art (see for example Greene et al, “Protecting groups in organic synthesis”, 2nd Edition, Wiley, New York, 1991, the disclosure of which is hereby incorporated by reference). Suitable protecting groups for any —OH groups in the carbohydrate moiety include acetate (Ac), benzyl (Bn), silyl (for example tert-butyl dimethylsilyl (TBDMSi) and tert-butyldiphenylsilyl (TMDPSi)), acetals, ketals, and methoxymethyl (MOM). Any protecting groups may be removed before or after attachment of the carbohydrate moiety to the peptide linker.

In a preferred embodiment of the invention said sugars are unprotected.

Particularly preferred carbohydrate moieties include Glc(Ac)₄β-, Glc(Bn)₄β-, Gal(Ac)₄β-, Gal(Bn)₄β-, Glc(Ac)₄α(1,4)Glc(Ac)₃α(1,4)Glc(Ac)₄β-, β-Glc, β-Gal, -Et-β-Gal,-Et-β-Glc, Et-α-Glc, -Et-α-Man, -Et-Lac, -β-Glc(Ac)₂, -β-Glc(Ac)₃, -Et-α-Glc(Ac)₂, -Et-α-Glc(Ac)₃, -Et-α-Glc(Ac)₄, -Et-β-Glc(Ac)₂, -Et-β-Glc(Ac)₃, -Et-β-Glc(Ac)₄, -Et-α-Man(Ac)₃, -Et-α-Man(Ac)₄, -Et-β-Gal(Ac)₃, -Et-β-Gal(Ac)₄, -Et-Lac(Ac)₅, -Et-La [symbol] and their deprotected equivalents.

Preferably, any saccharide units making up the carbohydrate moiety which are derived from naturally occurring sugars will each be in the, naturally occurring enantiomeric form, which may be either the D-form (e.g. D-glucose or D-galactose), or the L-form (e.g. L-rhamnose or L-fucose). Any anomeric linkages may be α- or β- linkages.

According to a further aspect of the invention said fusion polypeptide is encoded by a nucleic acid molecule selected from the group consisting of:

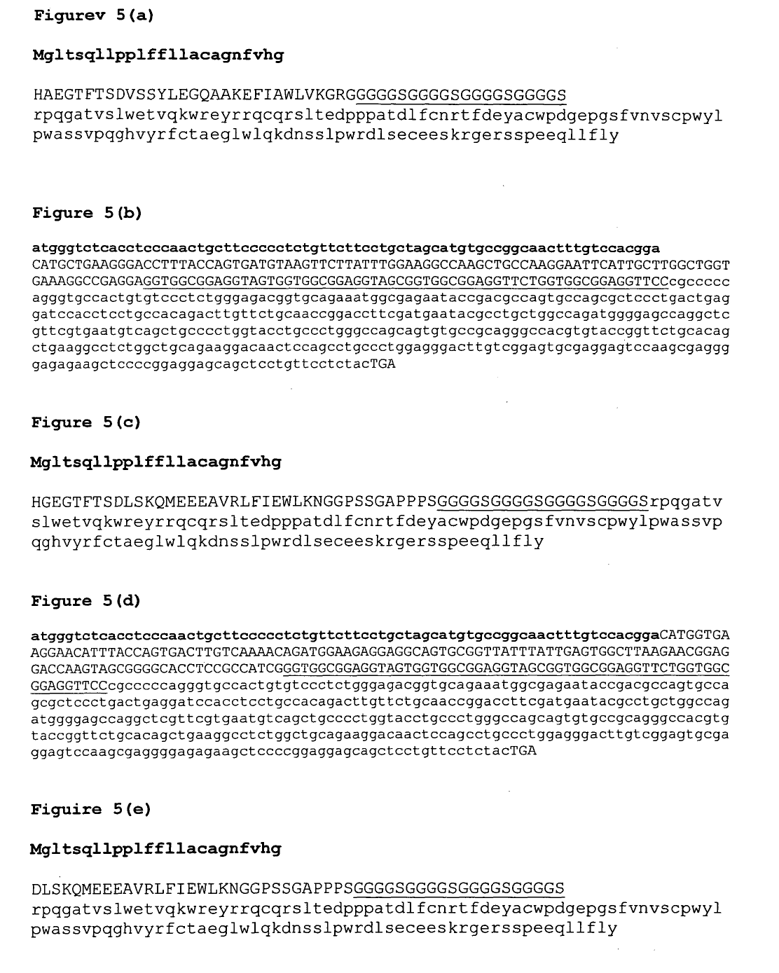

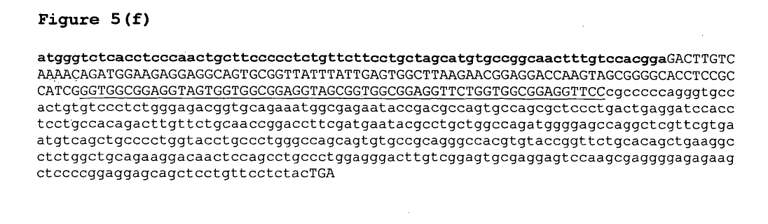

i) a nucleic acid sequence as represented in FIG. 5b;

ii) a nucleic acid sequence as represented in FIG. 5d;

iii) a nucleic acid sequence as represented in FIG. 5f;

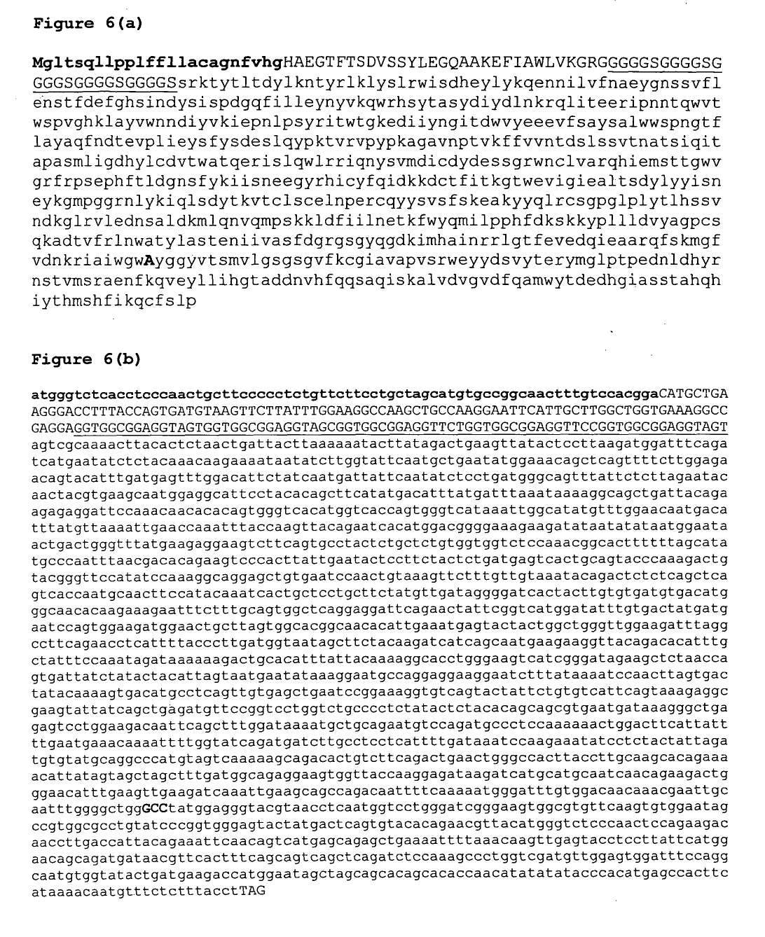

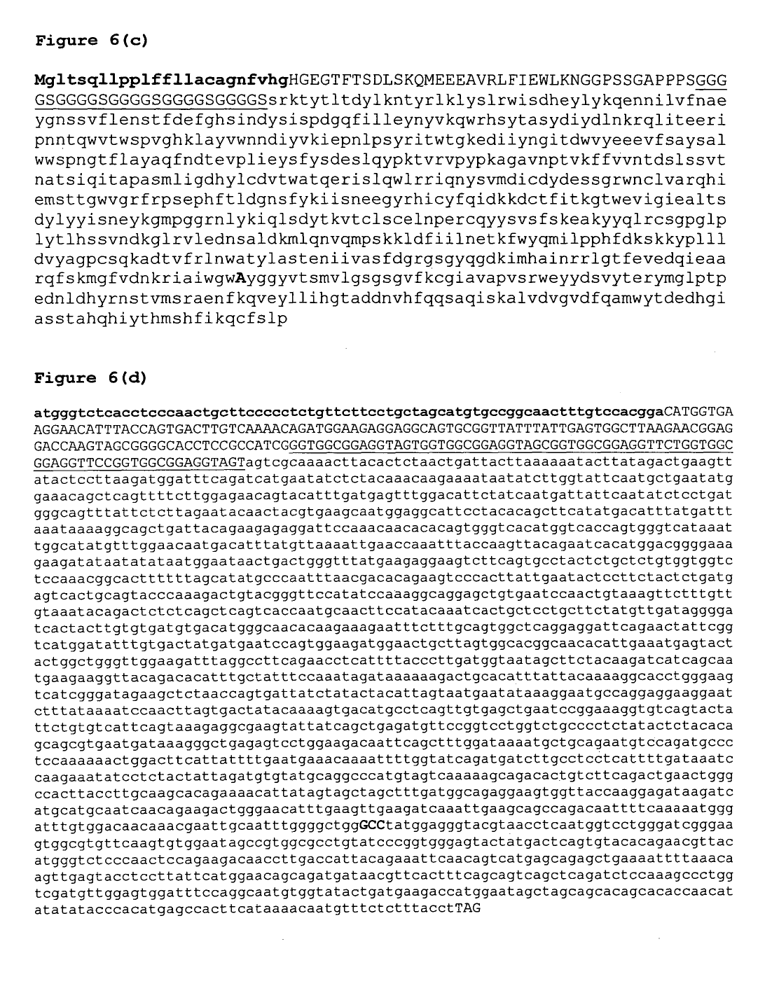

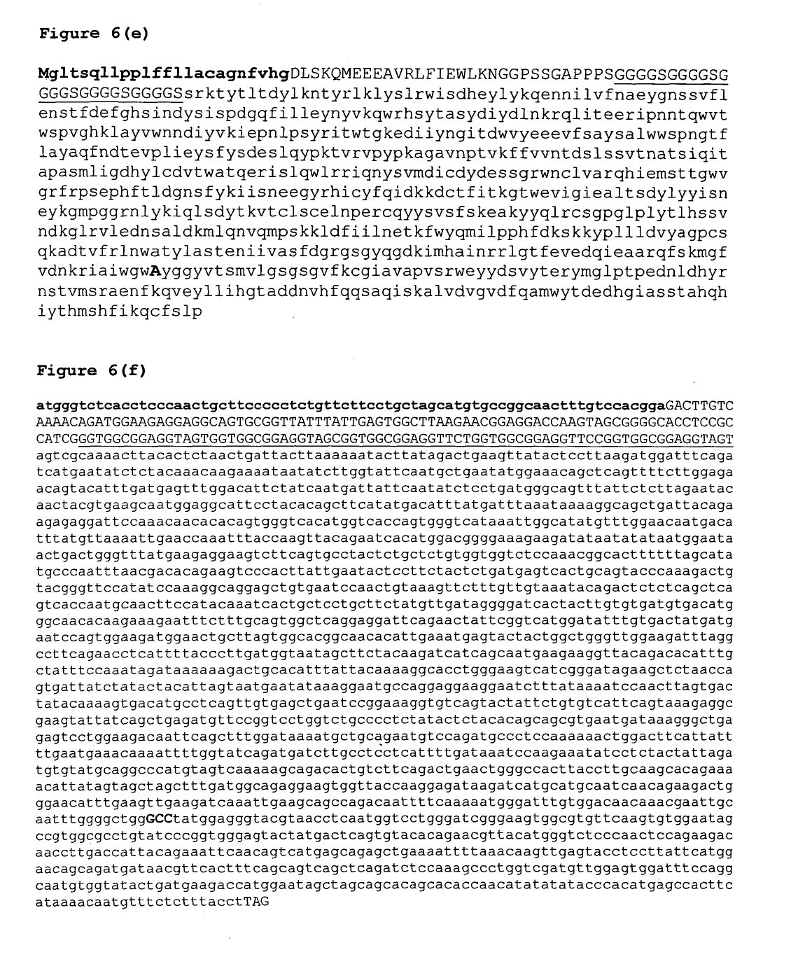

iv) a nucleic acid sequence as represented in FIG. 6b;

v) a nucleic acid sequence as represented in FIG. 6d;

vi) a nucleic acid sequence as represented in FIG. 6f;

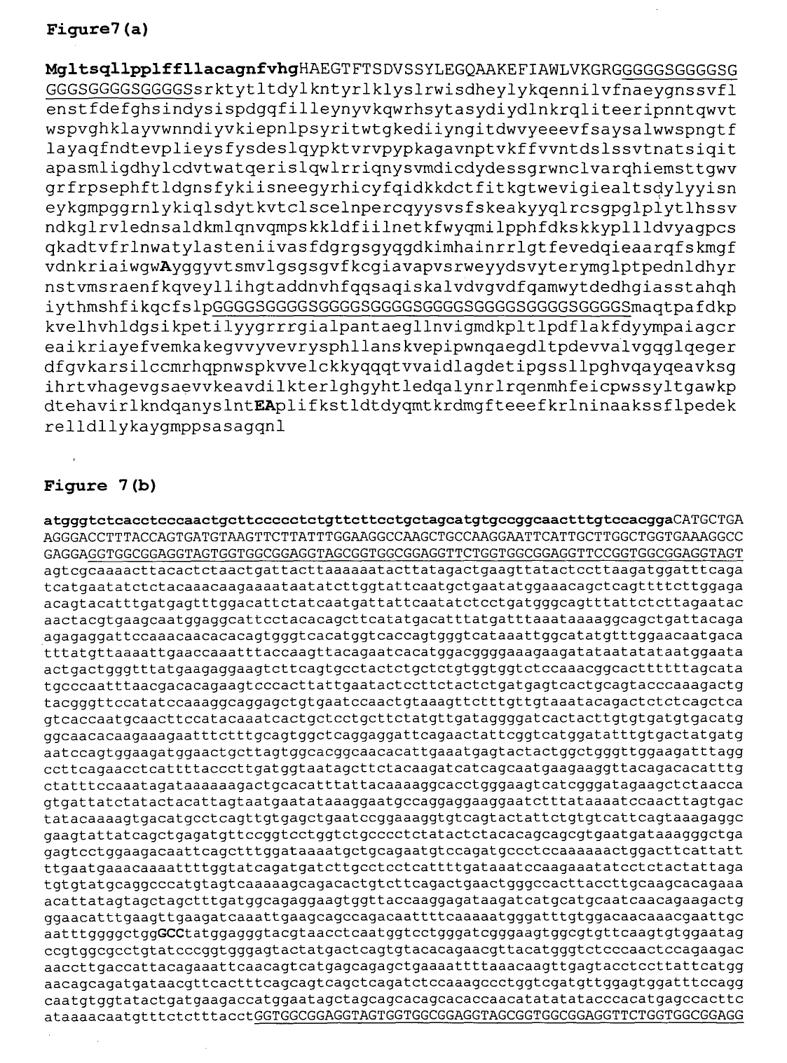

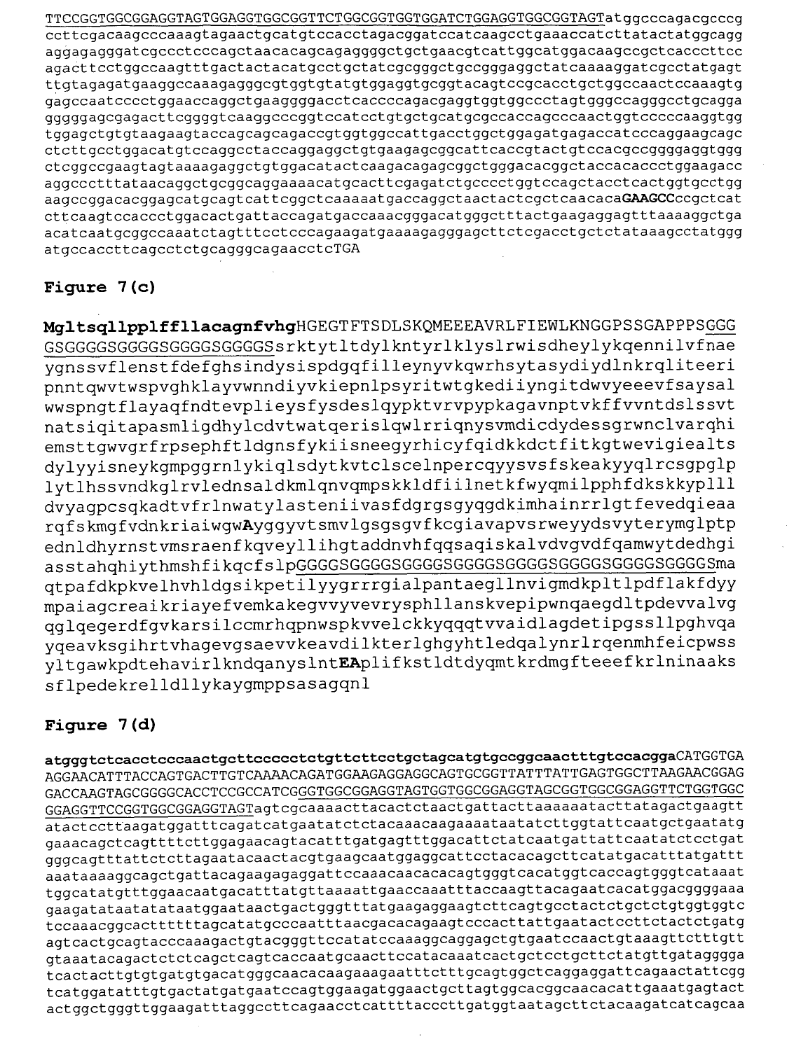

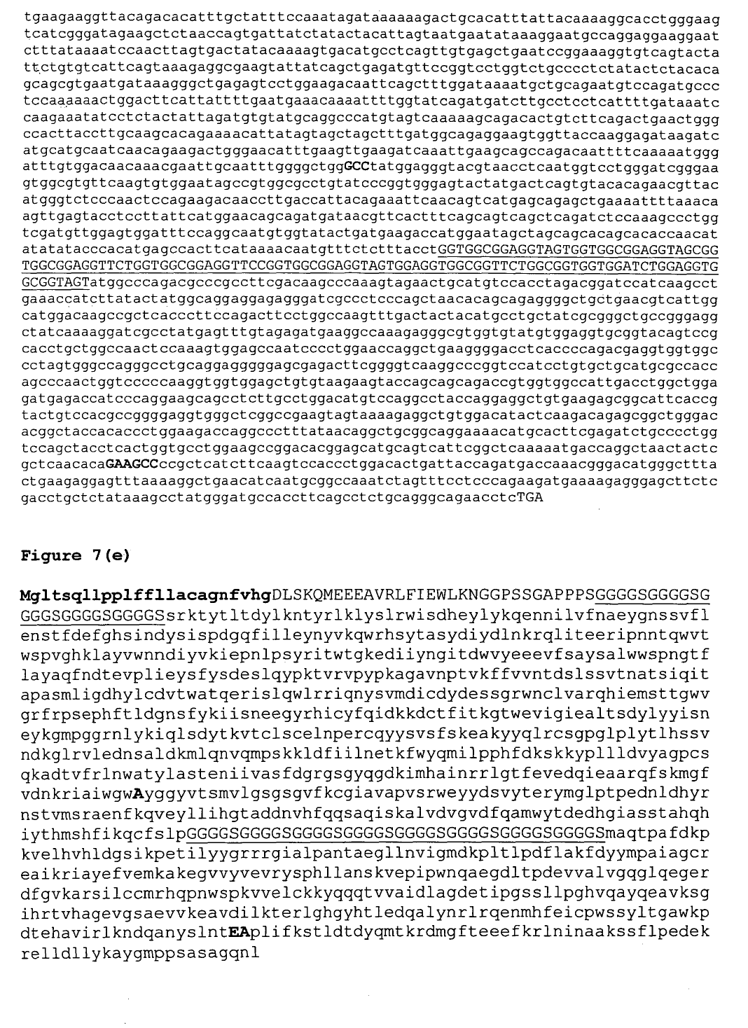

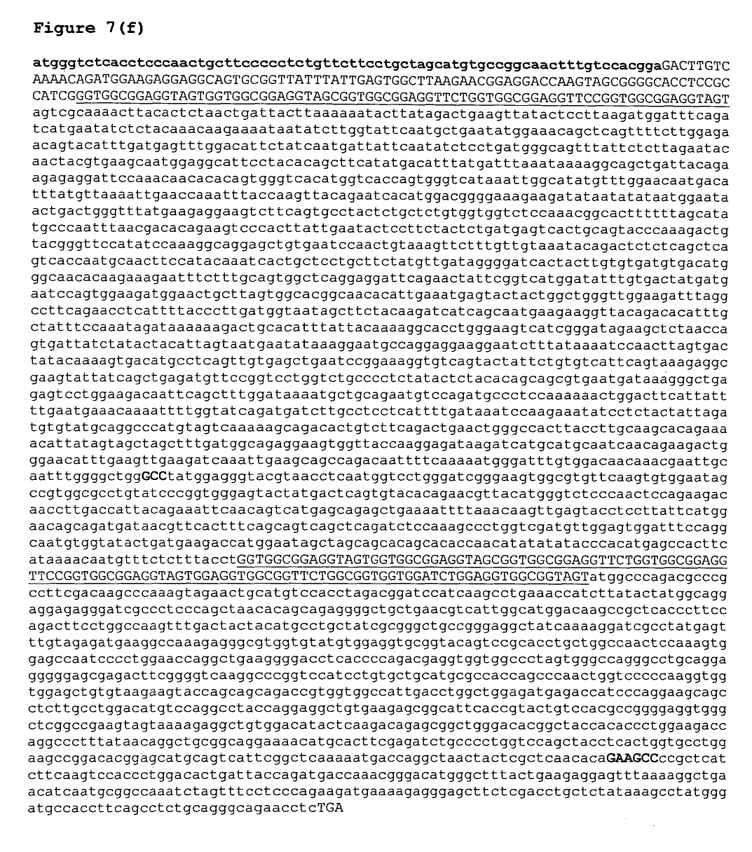

vii) a nucleic acid sequence as represented in FIG. 7b;

viii) a nucleic acid sequence as represented in FIG. 7d;

ix) a nucleic acid sequence as represented in FIG. 7f;

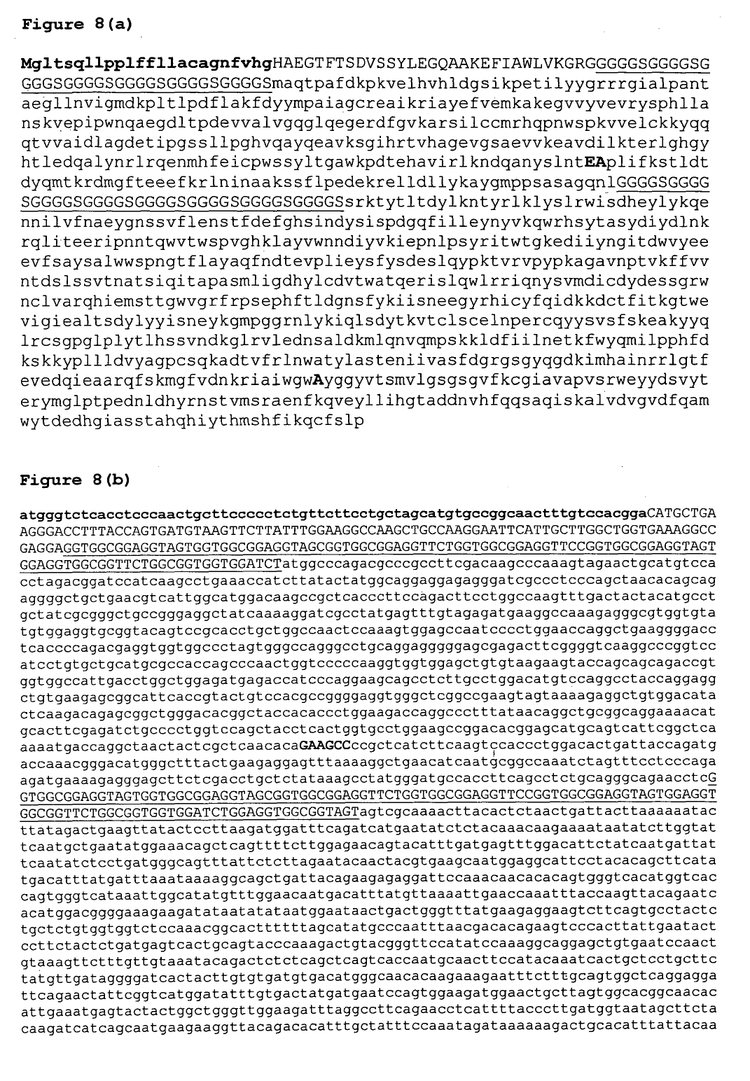

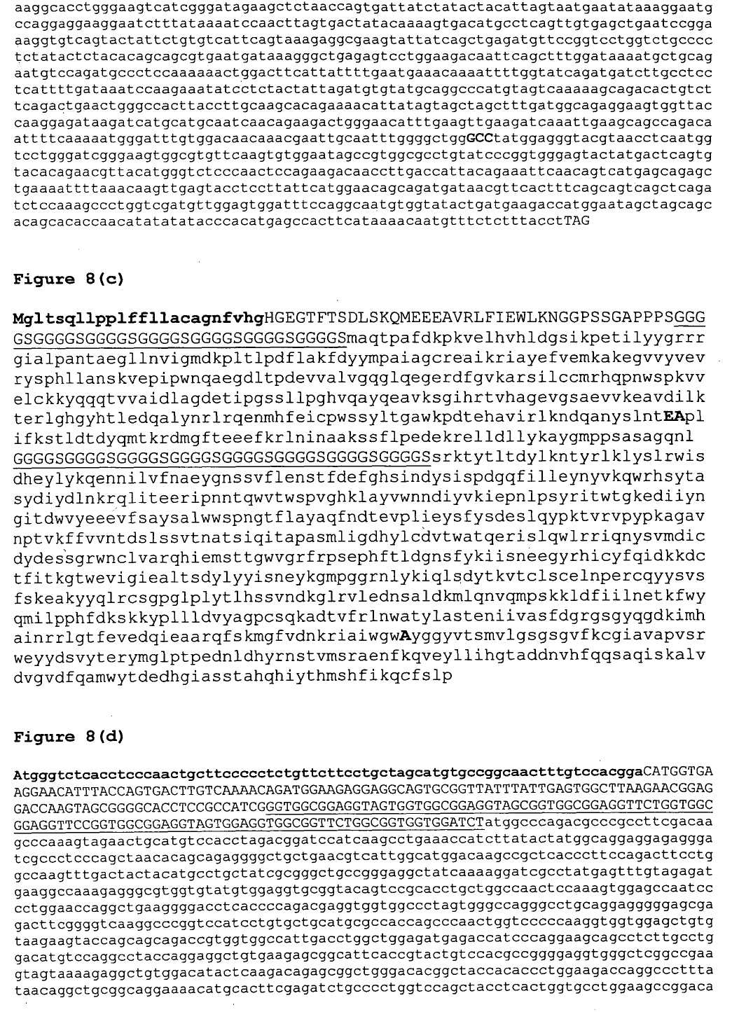

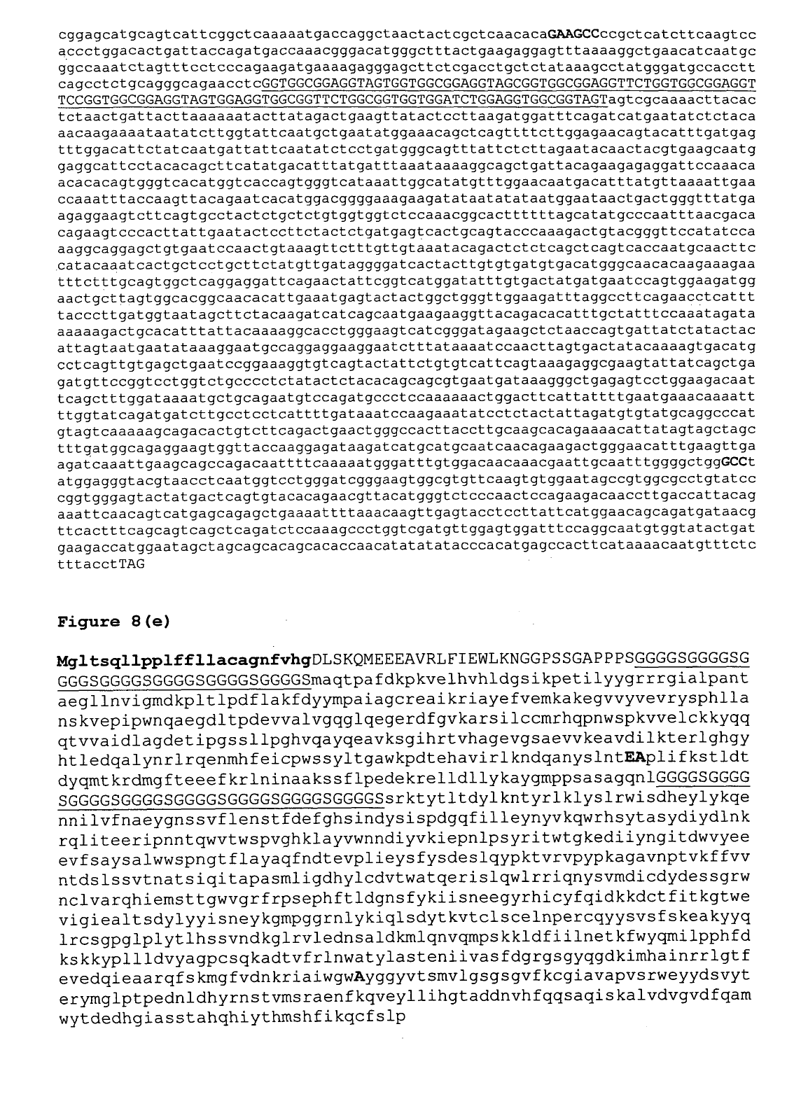

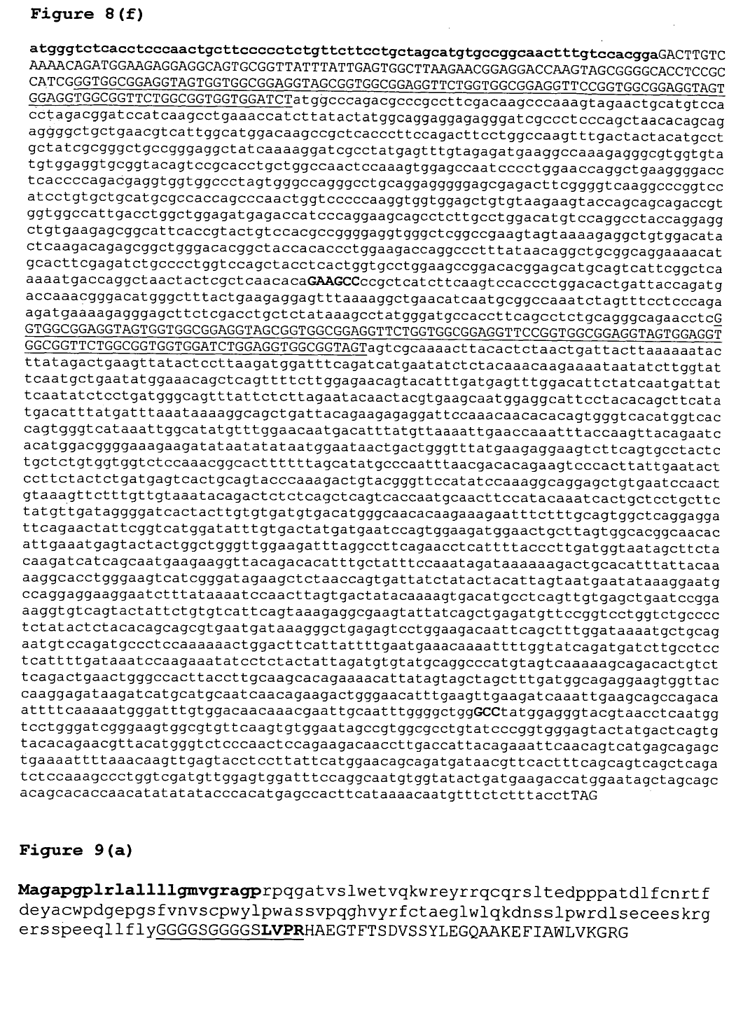

x) a nucleic acid sequence as represented in FIG. 8b;

xi) a nucleic acid sequence as represented in FIG. 8d;

xii) a nucleic acid sequence as represented in FIG. 8f;

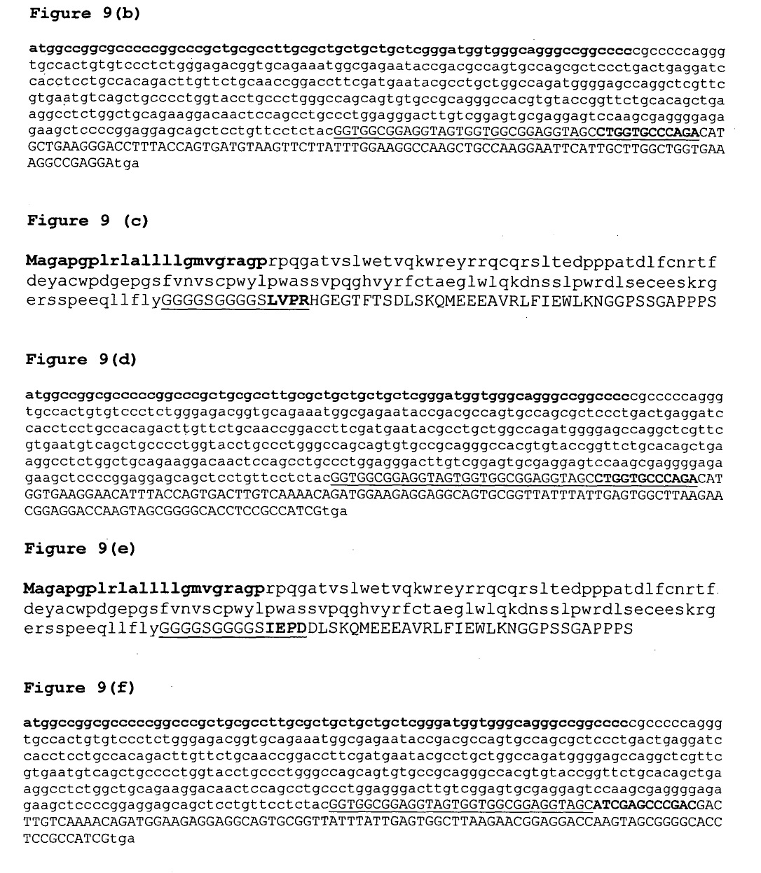

xiii) a nucleic acid sequence as represented in FIG. 9b;

xiv) a nucleic acid sequence as represented in FIG. 9d;

xv) a nucleic acid sequence as represented in FIG. 9f;

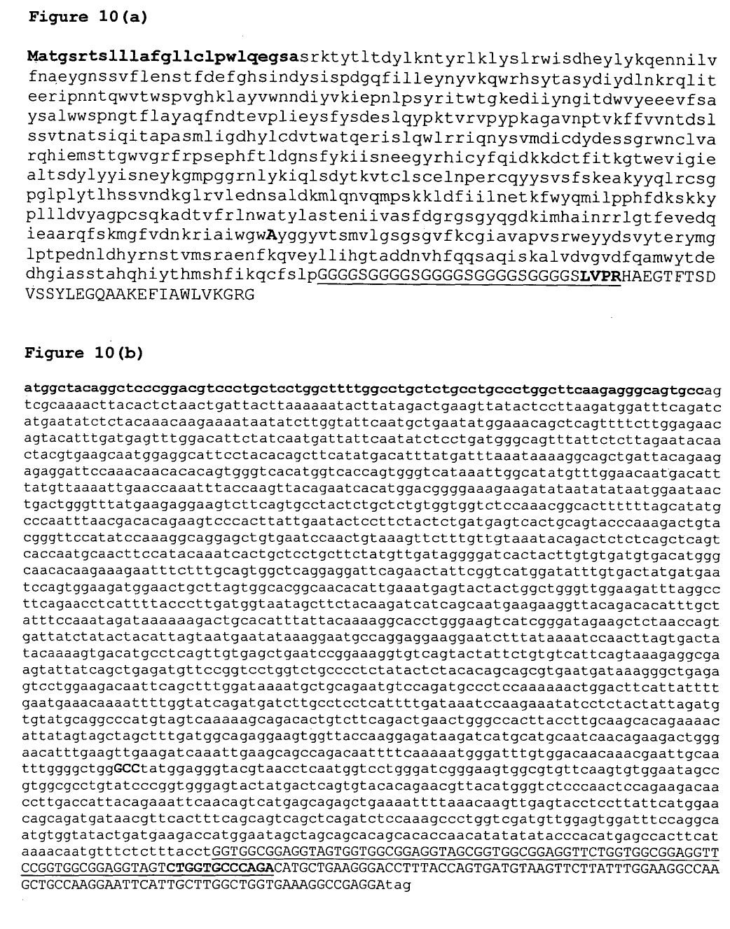

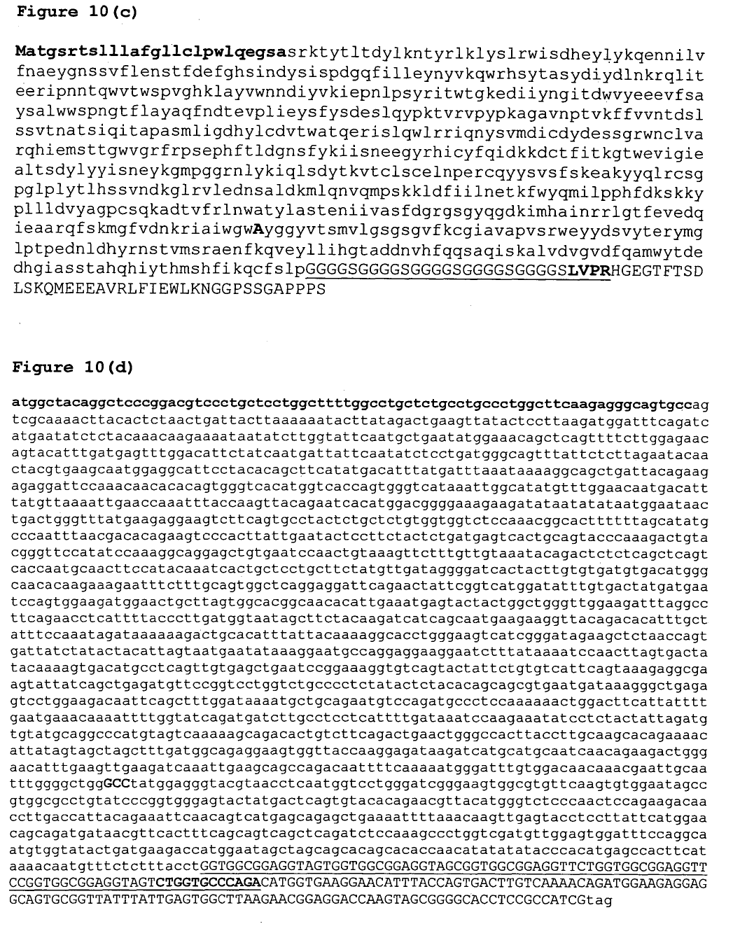

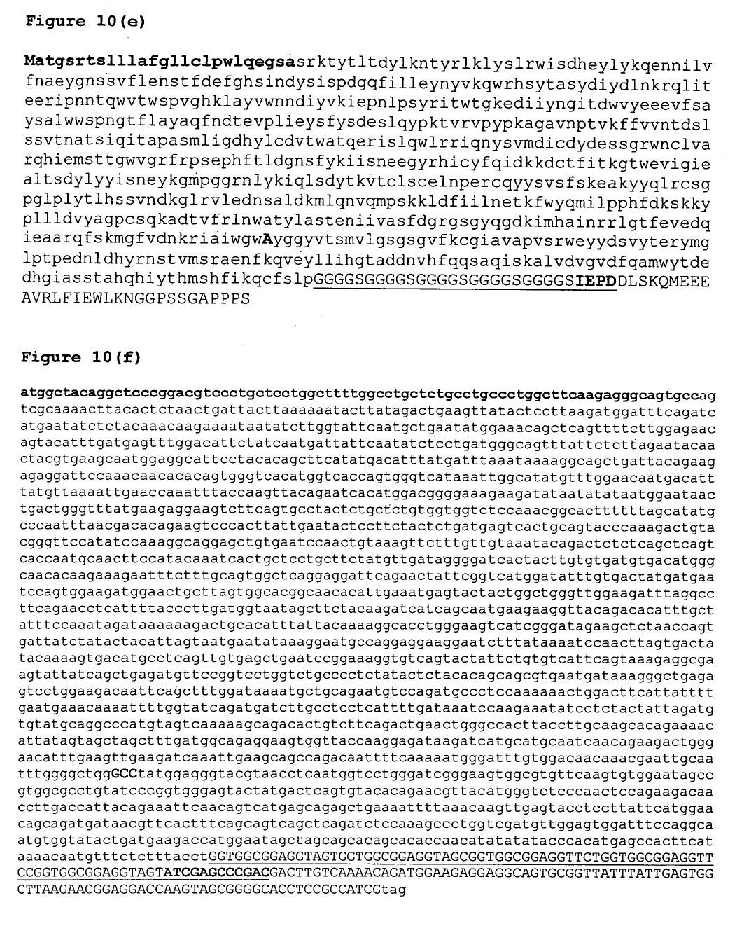

xvi) a nucleic acid sequence as represented in FIG. 10b;

xvii) a nucleic acid sequence as represented in FIG. 10d;

xviii) a nucleic acid sequence as represented in FIG. 10f;

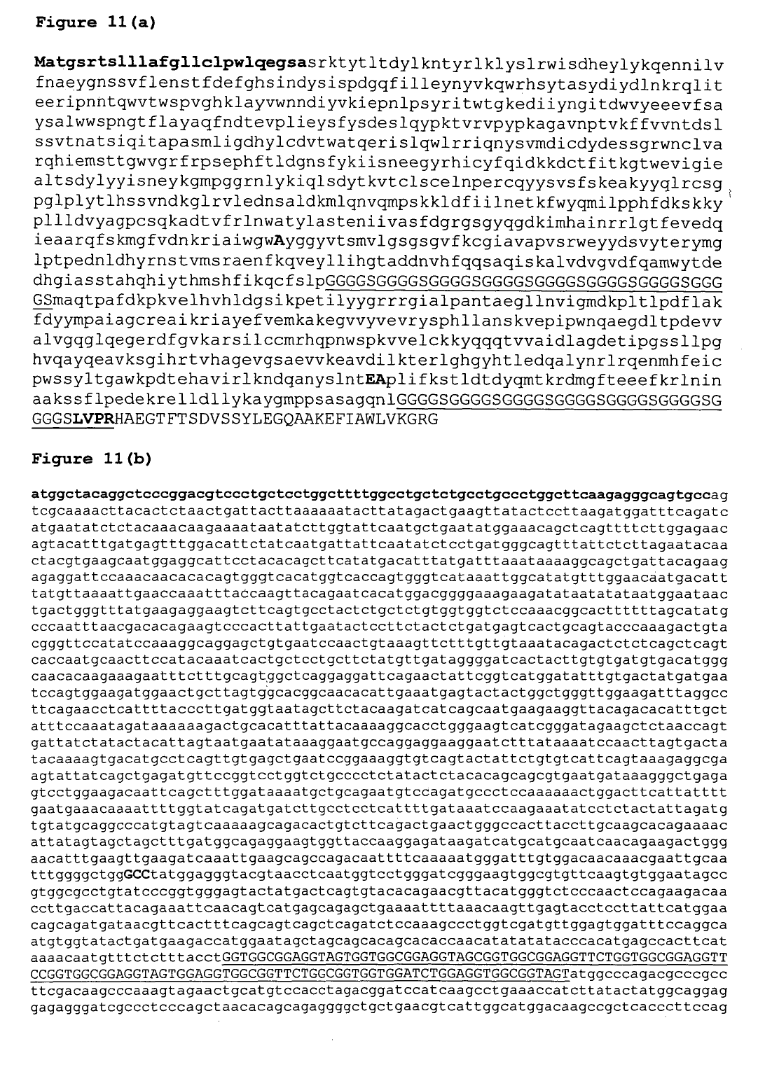

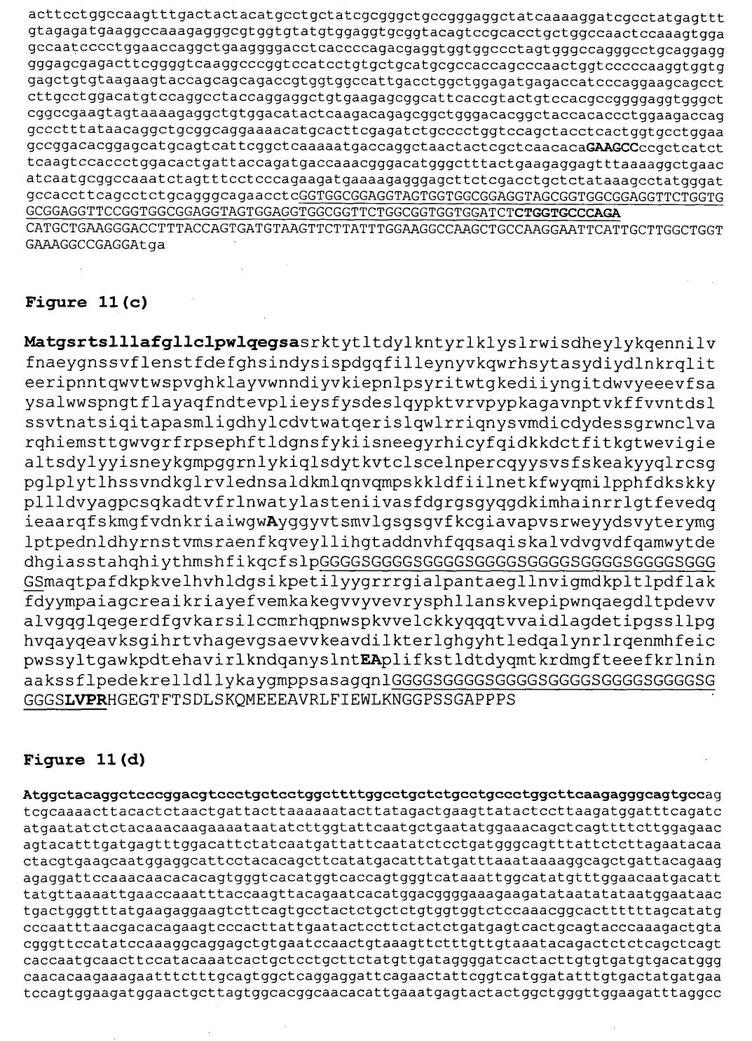

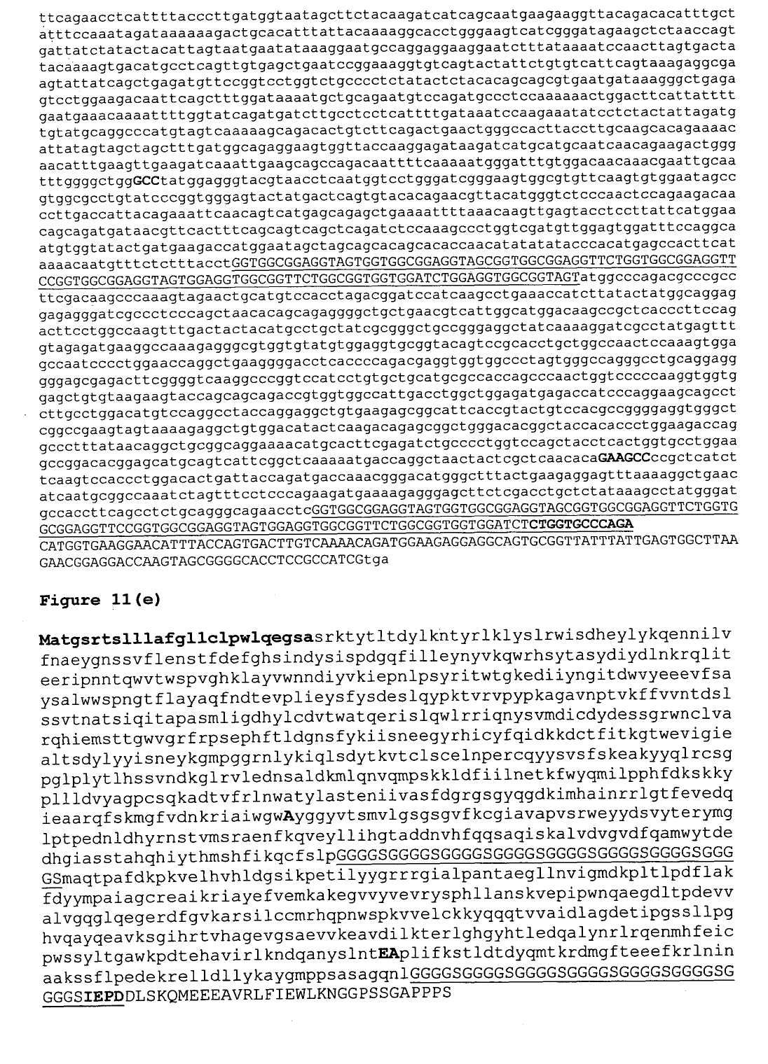

xix) a nucleic acid sequence as represented in FIG. 11b;

xx) a nucleic acid sequence as represented in FIG. 11d;

xxi) a nucleic acid sequence as represented in FIG. 11f;

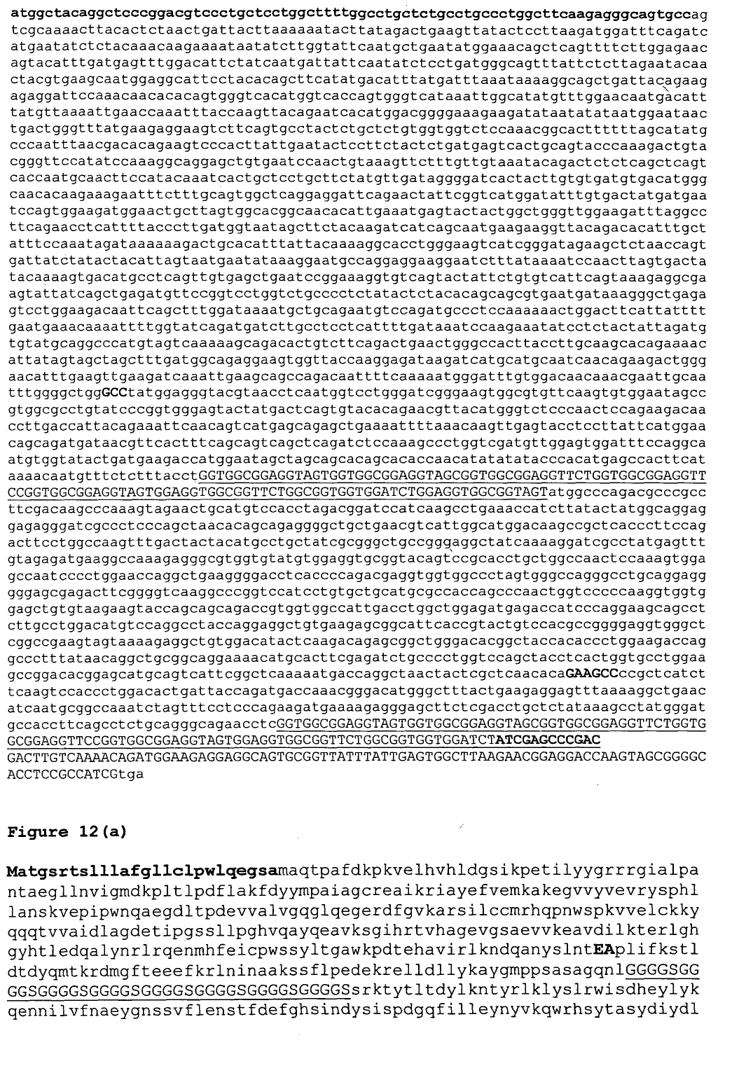

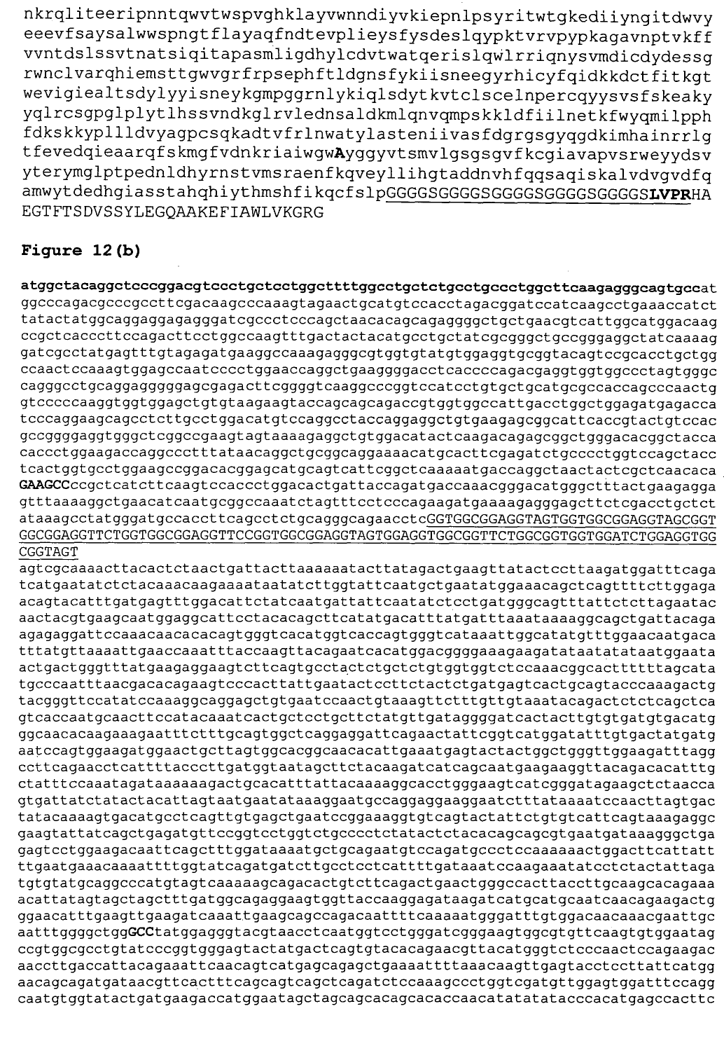

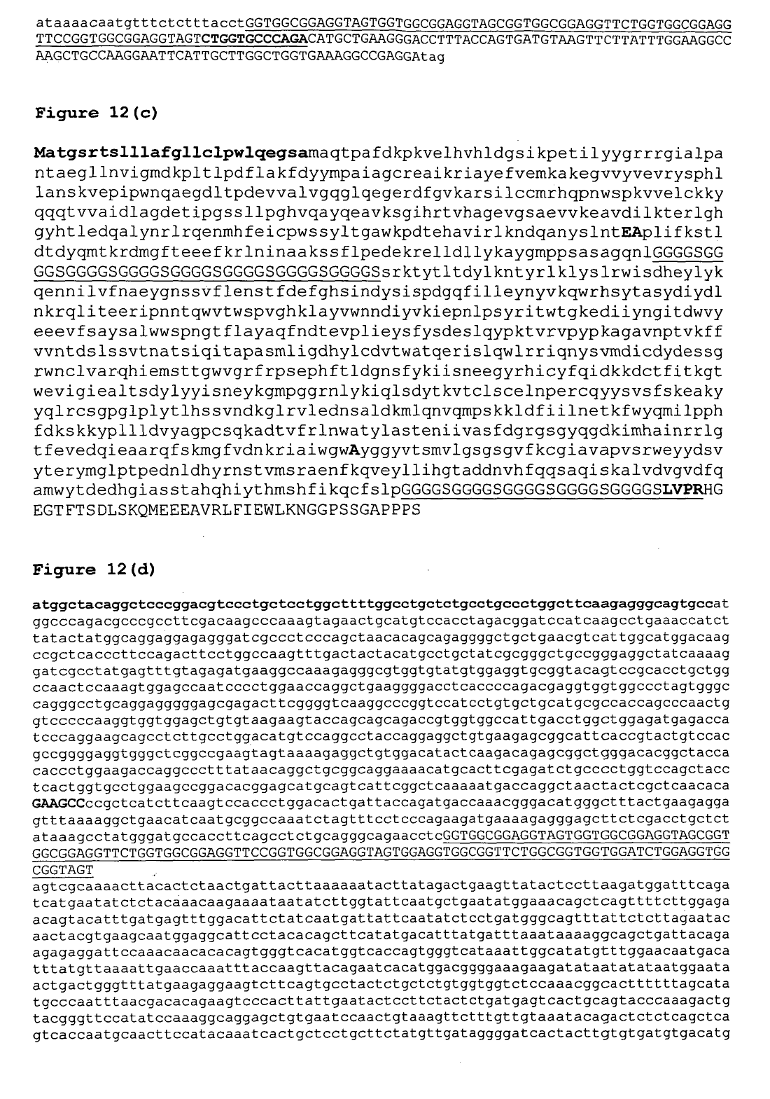

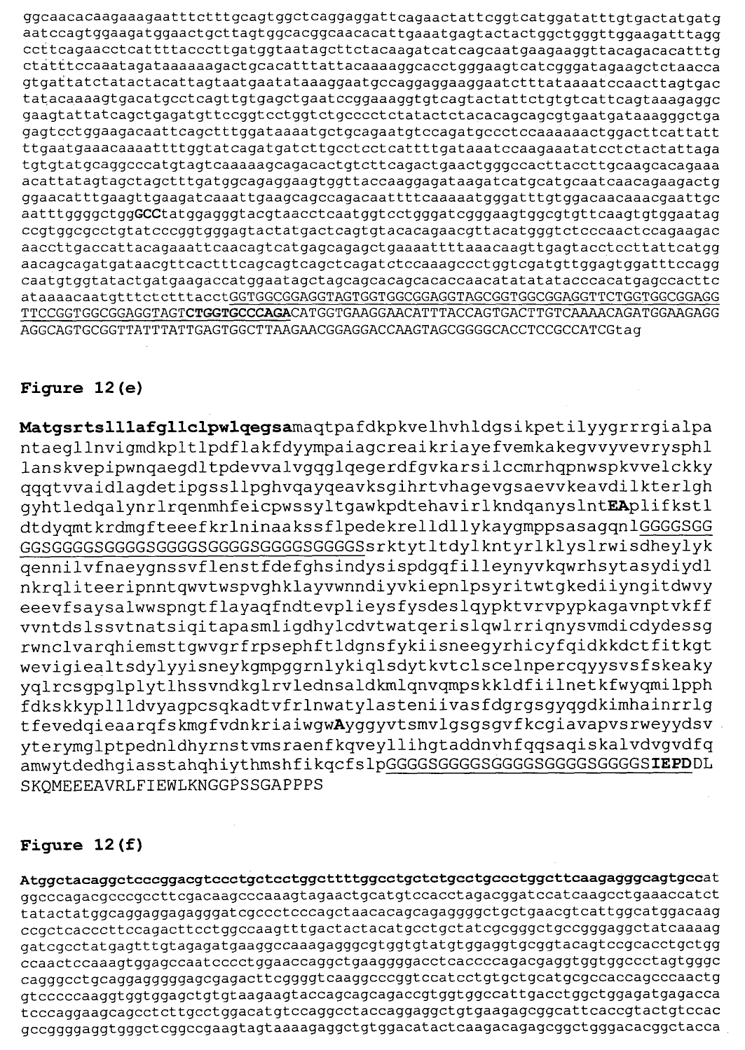

xxii) a nucleic acid sequence as represented in FIG. 12b;

xxiii) a nucleic acid sequence as represented in FIG. 12d;

xxiv) a nucleic acid sequence as represented in FIG. 12f; or a nucleic acid molecule comprising a nucleic sequence that hybridizes under stringent hybridization conditions to the nucleic acid sequence represented in FIG. 5b-12f and which encodes a polypeptide that has GLP-1 receptor modulating activity.

In a preferred embodiment of the invention said nucleic acid molecule encodes a polypeptide that has agonist activity.

There are a number of pathological conditions result in hyperglycaemia and would benefit from a GLP-1 agonist the most well known being diabetes mellitus. Diabetes mellitus can be of type 1 or type 2. Type 1 diabetes is an autoimmune disease resulting in destruction of the pancreatic β cells which means the subject is unable to manufacture any insulin. Type 2 diabetes is a more complicated condition and can result from a number of associated ailments but commonly involves resistance to the metabolic actions of insulin. For example, type 2 diabetes is associated with age, obesity, a sedentary life style which results in insulin resistance. An associated condition is called Metabolic Syndrome which may predispose subjects to type 2 diabetes. The symptoms associated with this syndrome are high blood pressure, dyslipidemia, increased body fat deposition and cardiovascular disease.

In an alternative preferred embodiment of the invention said nucleic acid molecule encodes a polypeptide that has antagonist activity.

Hypoglycaemia [abnormally low levels of serum glucose] is also known and is typically the result of administration of an insulin overdose. This would benefit from the administration of a GLP-1 antagonist. There are also diseases that result in excess insulin secretion resulting in a hypoglycaemic state. For example, insulinoma is a cancer of the pancreatic β cells resulting in over production of insulin. Other examples that may benefit from GLP-1 antagonism include hyperinsulinism, anorexia and controlling glucagon secretion in type 1 diabetes.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at least 90% Identity to Hybridize)

Hybridization: 5× SSC at 65° C. for 16 hours

Wash twice: 2× SSC at room temperature (RT) for 15 minutes each

Wash twice: 0.5× SSC at 65° C. for 20 minutes each

High Stringency (Allows Sequences that Share at least 80% Identity to Hybridize)

Hybridization: 5×-6× SSC at 65° C.-70° C. for 16-20 hours

Wash twice: 2× SSC at RT for 5-20 minutes each

Wash twice: 1× SSC at 55° C.-70° C. for 30 minutes each

Low Stringency (Allows Sequences that Share at least 50% Identity to Hybridize)

Hybridization: 6× SSC at RT to 55° C. for 16-20 hours

Wash at least twice: 2×-3× SSC at RT to 55° C. for 20-30 minutes each.

According to a further aspect of the invention there is provided a polypeptide encoded by a nucleic acid molecule according to the invention.

According to an aspect of the invention there is provided a polypeptide comprising an amino acid sequence selected from the group consisting of: FIG. 5a, 5c, 5e, 6a, 6c, 6e, 7a, 7c, 7e, 8a, 8c, 8e, 9a, 9c, 9e, 10a, 10c, 10e, 11a, 11c, 11e, 12a, 12c, 12e, 13a, 13c, 13e, 14a, 14c, 14e, 15a, 15c, 15e, 16a, 16c, 16e, 17a, 17c, 17e, 18a, 18c, 18e, 19a, 19c, 19e, 20a, 20c or 20e.

According to an aspect of the invention there is provided a homodimer consisting of two polypeptides according to the invention.

According to a further aspect of the invention there is provided a nucleic acid molecule that encodes a polypeptide according to the invention.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articular, subcutaneous, topical, dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceutical compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts [symbol] potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

According to a further aspect of the invention there is provided a method to treat a human subject suffering from hyperglycaemia comprising administering an effective amount of at least one polypeptide according to the invention.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

In a preferred method of the invention said hyperglycaemic condition is diabetes mellitus.

In a preferred method of the invention diabetes mellitus is type I.

In a preferred method of the invention diabetes mellitus is type II.

In a preferred method of the invention said hyperglycaemia is the result of insulin resistance.

In a preferred method of the invention said hyperglycaemia is the result of Metabolic Syndrome.

According to an aspect of the invention there is provided the use of a polypeptide according to the invention for the manufacture of a medicament for the treatment of diabetes mellitus.

In a preferred embodiment of the invention diabetes mellitus is type I.

In a preferred embodiment of the invention diabetes mellitus is type I

[symbol]

According to a further aspect of the invention there is provided a monoclonal antibody that binds the polypeptide or dimer according to the invention.

Preferably said monoclonal antibody is an antibody that binds the polypeptide or dimer but does not specifically bind GLP-1 or GLP-1 receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and

Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1a is the nucleic acid sequence and amino acid sequence of human GLP-1 and human GLP-1 precursor; FIG. 1b is the amino acid sequence of exendin 4 precursor; FIG. 1c is the amino acid sequence of GLP-1 (7-37); FIG. 1d is the amino acid sequence of GLP-1 (7-36); FIG. 1e is the amino acid sequence of exendin-4; FIG. 1f is the amino acid sequence of exendin 4(9-39);

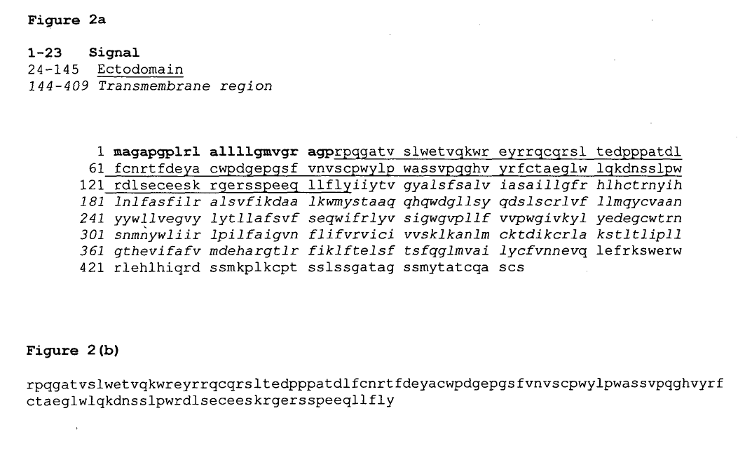

FIG. 2a is the full length amino acid sequence of human GLP-1 receptor; FIG. 2b is the amino acid sequence of the GLP-1 ectodomain;

FIG. 3a is the amino acid sequence of human DPP4; FIG. 3b is the amino acid sequence of inactive DPP4;

FIG. 4a is the amino acid sequence of human ADA; Figure b is the amino acid sequence of inactive ADA;

FIG. 5a is the full length amino acid sequence of IL4ss-GLP1-(G4S)4-GLP1R(24-145) fusion polypeptide and FIG. 5b is the nucleic acid sequence; FIG. 5c is the full length amino acid sequence of IL4ss-exendin-(G4S)4-GLP1R(24-145) fusion polypeptide and FIG. 5d is the nucleic acid sequence; FIG. 5e is the full length amino acid sequence of IL4ss-Ex4(9-39)-(G4S)4-GLP1R(24-145) antagonist fusion polypeptide and FIG. 5f is the nucleic acid sequence;

FIG. 6a is the full length amino acid sequence of IL4ss-GLP1-(G4S)5-DPP4(39-766; S630A) fusion polypeptide and FIG. 6b is the nucleic acid sequence; FIG. 6c is the full length amino acid sequence of IL4ss-exendin-(G4S)5-DPP4(39-766; S630A) fusion polypeptide and FIG. 6d is the nucleic acid sequence; FIG. 6e is the full length amino acid sequence of IL4ss-Ex4(9-39)-(G4S)5-DPP4(39-766; S630A) antagonist fusion polypeptide and FIG. 6f is the nucleic acid sequence;

FIG. 7a is the full length amino acid sequence of IL4ss-GLP1-(G4S)5-DPP4(39-766; S630A)-(G4S)8-ADA D295E, D296A) fusion polypeptide and FIG. 7b is the nucleic acid sequence; FIG. 7c is the full length amino acid sequence of IL4ss-exendin-(G4S)5-DPP4(39-766; S630A)-(G4S)8-ADA D295E, D296A) fusion polypeptide and FIG. 7d is the nucleic acid sequence; FIG. 7e is the full length amino acid sequence of IL4ss-Ex4(9-39)-(G4S)5-DPP4(39-766; S630A)-(G4S)8-ADA D295E, D296A) antagonist fusion polypeptide and FIG. 7f is the nucleic acid sequence;

FIG. 8a is the full length amino acid sequence of IL4ss-GLP1-(G4S)7-ADA D295E, D296A)-(G4S)8-DPP4(39-766; S630A) fusion polypeptide and FIG. 8b is the nucleic acid sequence; FIG. 8c is the full length amino acid sequence of IL4ss-exendin-(G4S)7-ADA D295E, D296A)-(G4S)8-DPP4(39-766; S630A) fusion polypeptide and FIG. 8d is the nucleic acid sequence; Figure [symbol] amino acid sequence of IL4ss-Ex4(9-39)-(G4S)7-ADA D295E, D296A)-(G4S) 8-DPP4(39-766; S630A) antagonist fusion polypeptide and FIG. 8f is the nucleic acid sequence;

FIG. 9a is the full length amino acid sequence of GLP1Rss-GLP1R(24-145) -(G4S)2 -LVPR- GLP1 fusion polypeptide and FIG. 9b is the nucleic acid sequence; FIG. 9c is the full length amino acid sequence of GLP1Rss-GLP1R(24-145) -(G4S)2-exendin fusion polypeptide and FIG. 9d is the nucleic acid sequence; FIG. 9e is the full length amino acid sequence of GLP1Rss-GLP1R(24-145)-(G4S)4-IEPD-Ex4(9-39) antagonist fusion polypeptide and FIG. 9f is the nucleic acid sequence;

FIG. 10a is the full length amino acid sequence of HGHss-DPP4(39-766; S630A) -(G4S)5-LVPR-GLP1 fusion polypeptide and FIG. 10b is the nucleic acid sequence; FIG. 10c is the full length amino acid sequence HGHss-DPP4(39-766; S630A) -(G4S)5-LVPR-exendin fusion polypeptide and FIG. 10d is the nucleic acid sequence; FIG. 10e is the full length amino acid sequence of HGHss-DPP4(39-766; S630A)-(G4S)5-IEPD-Ex4(9-39) antagonist fusion polypeptide and FIG. 10f is the nucleic acid sequence;

FIG. 11a is the full length amino acid sequence of HGHss-DPP4 (39-766; S630A) -(G4S)8-ADA D295E, D296A)-(G4S)7-GLP1 fusion polypeptide and FIG. 11b is the nucleic acid sequence; FIG. 11c is the full length amino acid sequence of HGHss-DPP4(39-766; S630A)-(G4S)8-ADA D295E, D296A)-(G4S)7-LVPR-exendin fusion polypeptide and FIG. 11d is the nucleic acid sequence; FIG. 11e is the amino acid sequence of full length HGHss-DPP4(39-766; S630A)-(G4S)8-ADA D295E, D296A)-(G4S)7-IEPD-Ex4(9-39) antagonist fusion polypeptide and FIG. 11f is the nucleic acid sequence;

FIG. 12a is the full length amino acid sequence of HGHss-ADA D295E, D296A) -(G4S)8-DPP4(39-766; S630A)-(G4S)5-LVPR-GLP1 fusion polypeptide and FIG. 12b; FIG. 12c HGHss-ADA D295E, D296A)-(G4S)8-DPP4(39-766; S630A) -(G4S)5-LVPR-exendin fusion polypeptide and FIG. 12d is the nucleic acid sequence; FIG. 12e is the full length amino acid sequence of HGHss-ADA D295E, D296A)-(G4S)8-DPP4(39-766; S630A)-(G4S)5-IEPD-E [symbol] fusion polypeptide and FIG. 12f is the nucleic acid sequence;

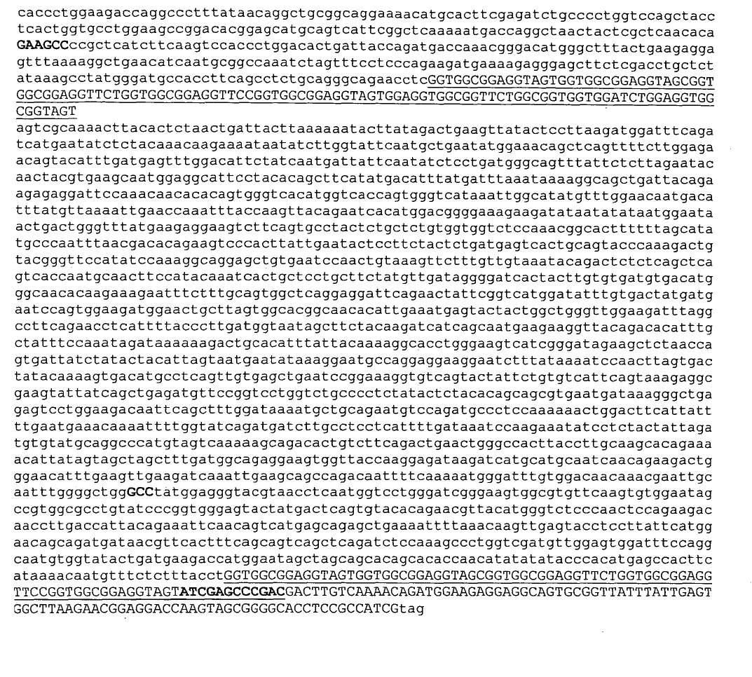

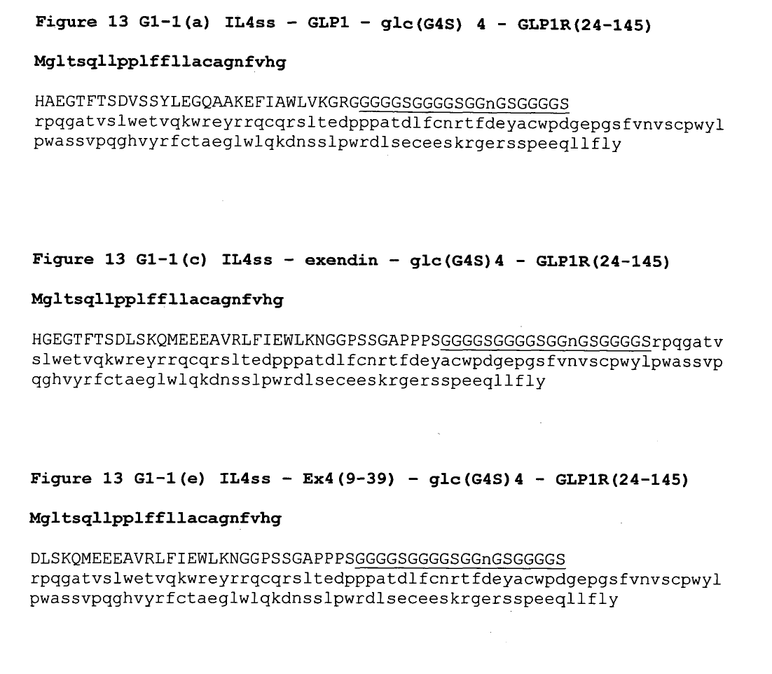

FIG. 13a is the full length amino acid sequence of IL4ss-GLP1-(G4S)4-GLP1R (24-145) fusion polypeptide; FIG. 13c is the full length amino acid sequence of IL4ss -exendin-(G4S)4-GLP1R(24-145) fusion polypeptide; FIG. 13e is the full length amino acid sequence of IL4ss-Ex4(9-39)-(G4S)4-GLP1R(24-145) antagonist fusion polypeptide each of which includes a peptide linker capable of glycosylation;

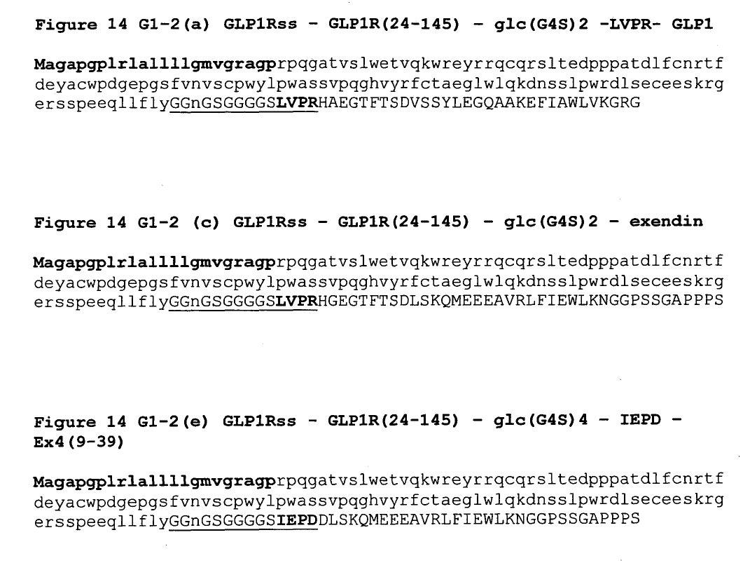

FIG. 14a is the full length amino acid sequence of GLP1Rss-GLP1R(24-145) -(G4S)2-LVPR-GLP1; FIG. 14c is the full length amino acid sequence of GLP1Rss-GLP1R(24-145)-(G4S)2-exendin fusion polypeptide; FIG. 14e is the full length amino acid sequence of GLP1Rss-GLP1R(24-145)-(G4S)4-IEPD-Ex4(9-39) antagonist fusion polypeptide;

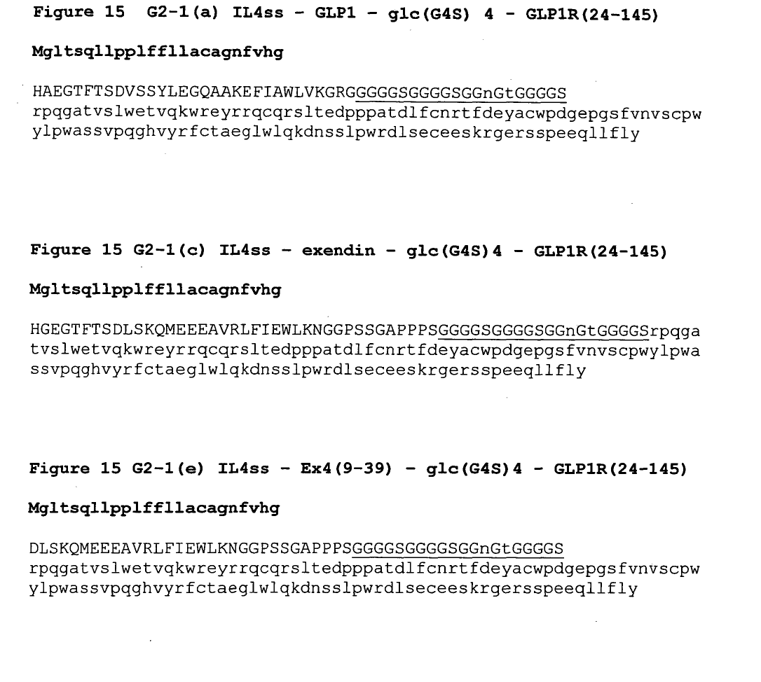

FIG. 15a is the full length amino acid sequence of IL4ss-GLP1-(G4S)4-GLP1R (24-145) fusion polypeptide; FIG. 15c is the full length amino acid sequence of IL4ss -exendin-(G4S)4-GLP1R(24-145) fusion polypeptide; FIG. 15e is the full length amino acid sequence of IL4ss-Ex4(9-39)-(G4S)4-GLP1R(24-145) antagonist fusion polypeptide each of which includes a peptide linker capable of glycosylation;

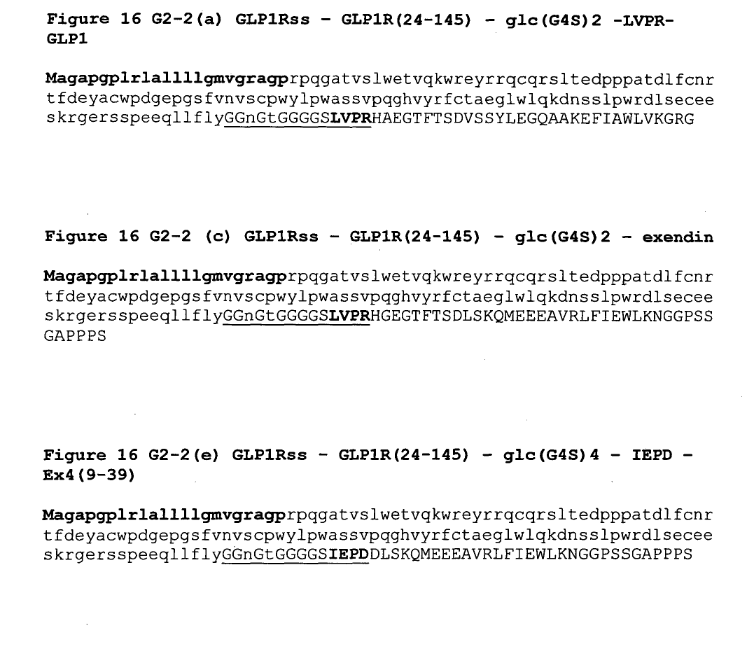

FIG. 16a is the full length amino acid sequence of GLP1Rss-GLP1R(24-145) -(G4S)2-LVPR-GLP1; FIG. 16c is the full length amino acid sequence of GLP1Rss -GLP1R(24-145)-(G4S)2-exendin fusion polypeptide; FIG. 16e is the full length amino acid sequence of GLP1Rss-GLP1R(24-145)-(G4S)4-IEPD-Ex4(9-39) antagonist fusion polypeptide;

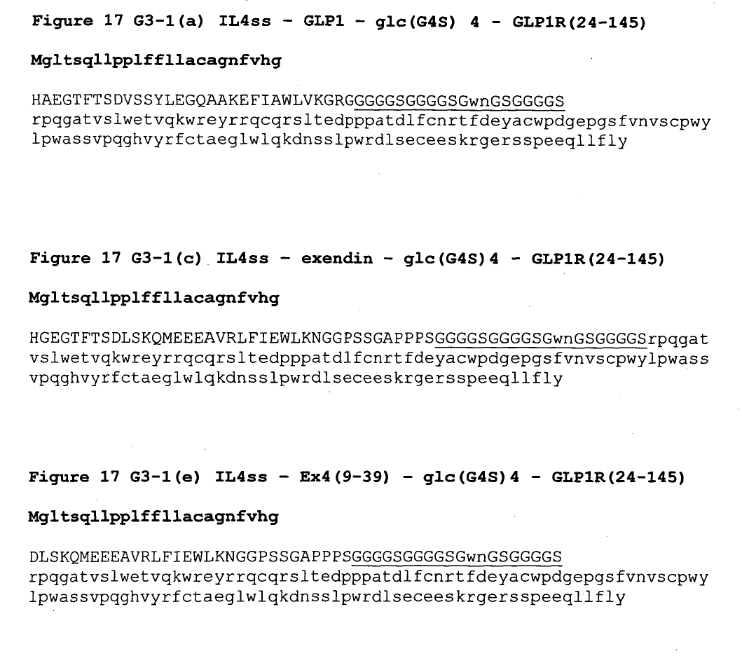

FIG. 17a is the full length amino acid sequence of IL4ss-GLP1-(G4S)4- GLP1R (24-145) fusion polypeptide; FIG. 17c is the full length amino acid sequence of IL4ss -exendin-G4S)4-GLP1R(24-145) fusion polypeptide; FIG. 17e is the full length amino acid sequence of IL4ss-Ex4(9-39)-(G4S)4-GLP1R(24-145) antagonist fusion polypeptide each of which includes a peptide linker capable of glycosylation;

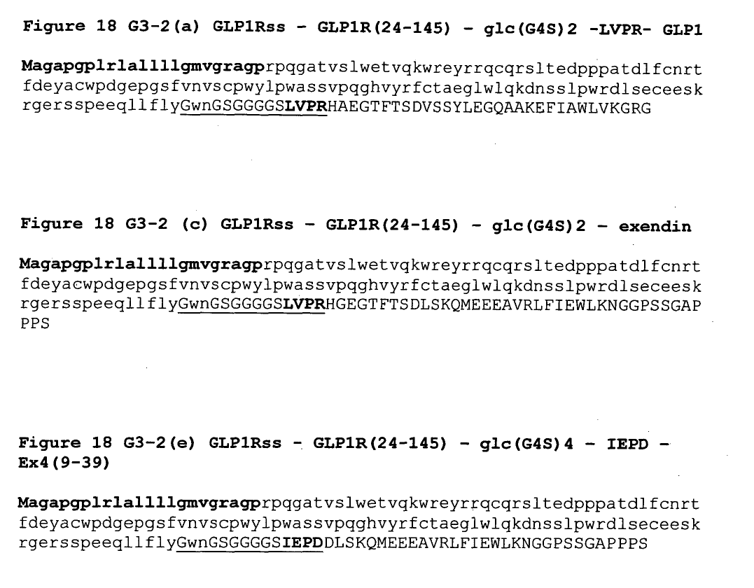

FIG. 18a is the full length amino acid sequence of GLP1Rss-GLP1R(24-145) -(G4S)2 -LVPR-GLP1; FIG. 18c is the full length amino acid sequence of GLP1Rss -GLP1R(24-145)-(G4S)2-exendin fusion polypeptide; FIG. 18e is the full length amino acid sequence of GLP1Rss-GLP1R(24-145)-(G4S)4 [symbol] -antagonist fusion polypeptide;

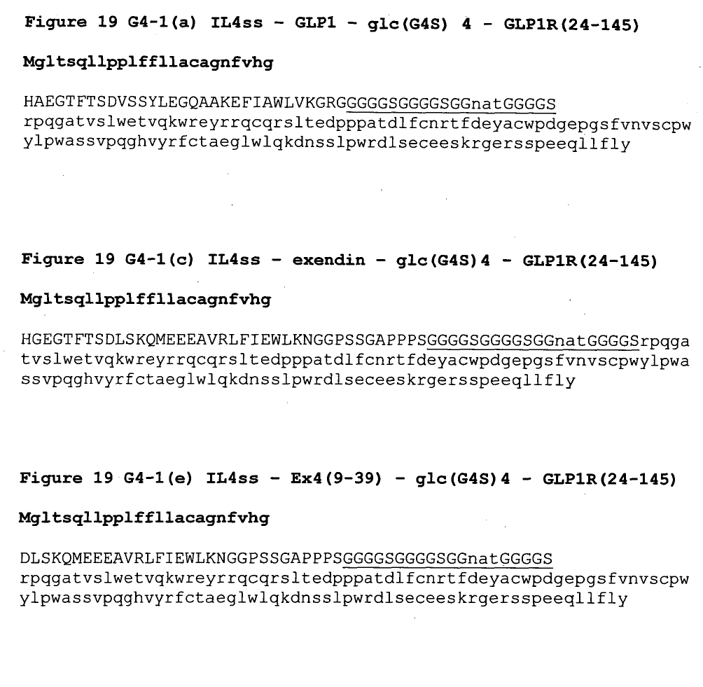

FIG. 19a is the full length amino acid sequence of IL4ss-GLP1-(G4S)4-GLP1R (24-145) fusion polypeptide; FIG. 19c is the full length amino acid sequence of IL4ss -exendin-(G4S)4-GLP1R(24-145) fusion polypeptide; FIG. 19e is the full length amino acid sequence of IL4ss-Ex4(9-39)-(G4S)4-GLP1 R(24-145) antagonist fusion polypeptide each of which includes a peptide linker capable of glycosylation;

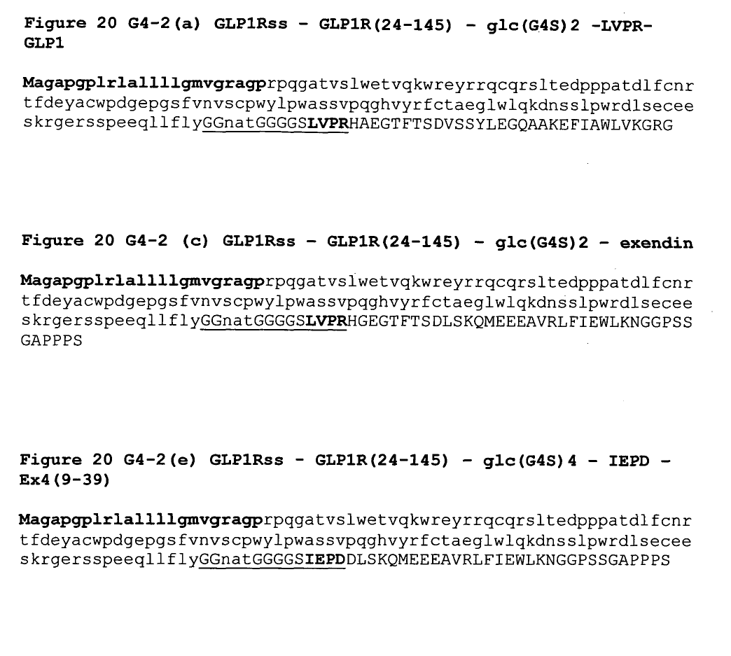

FIG. 20a is the full length amino acid sequence of GLP1Rss-GLP1R(24-145) -(G4S)2 -LVPR-GLP1; FIG. 20c is the full length amino acid sequence of GLP1Rss -GLP1R(24-145)-(G4S)2-exendin fusion polypeptide; FIG. 20e is the full length amino acid sequence of GLP1Rss-GLP1R(24-145)-(G4S)4-IEPD-Ex4 (9-39) antagonist fusion polypeptide;

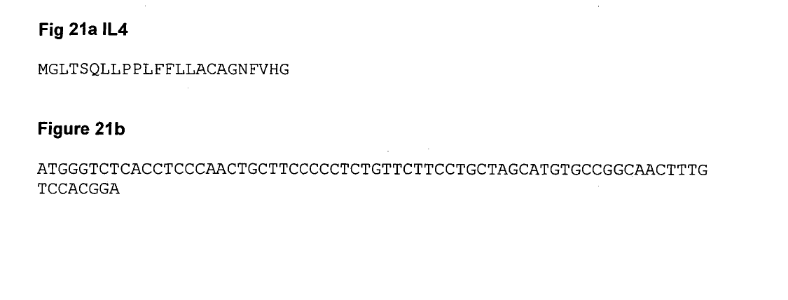

FIG. 21a is the nucleic acid sequence of the IL4 signal sequence; FIG. 21b is the amino acid sequence;

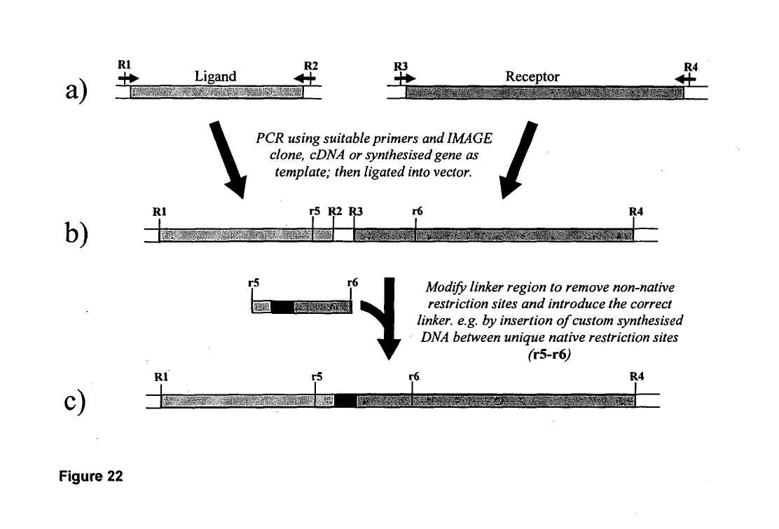

FIG. 22 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites); and

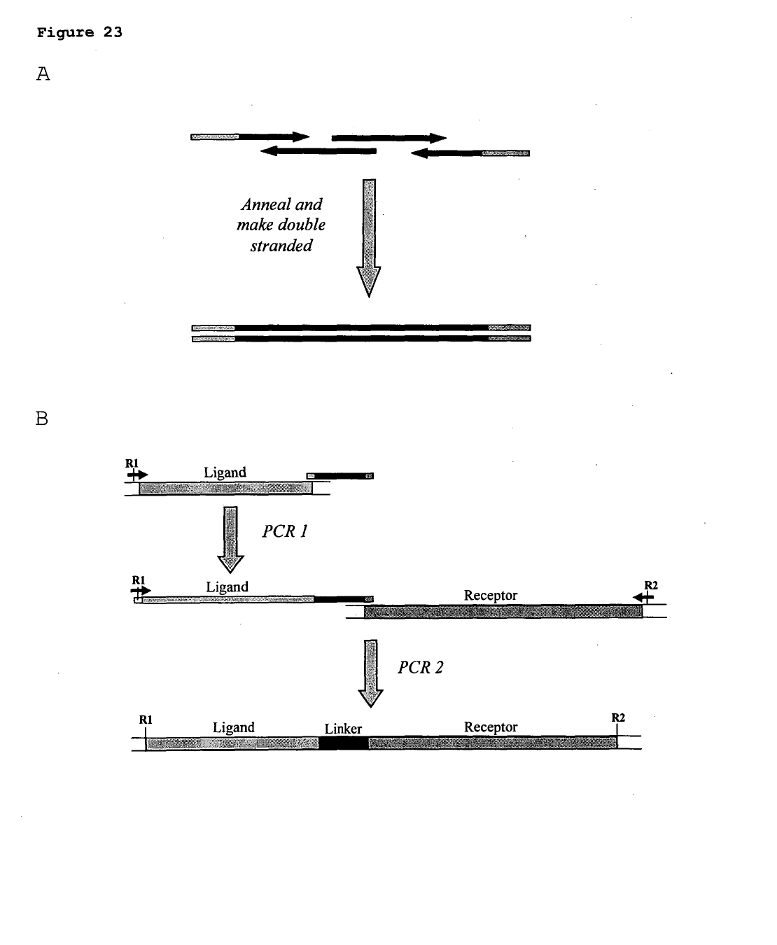

FIG. 23 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

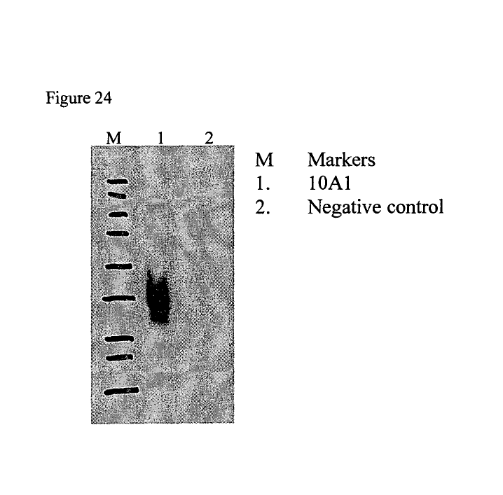

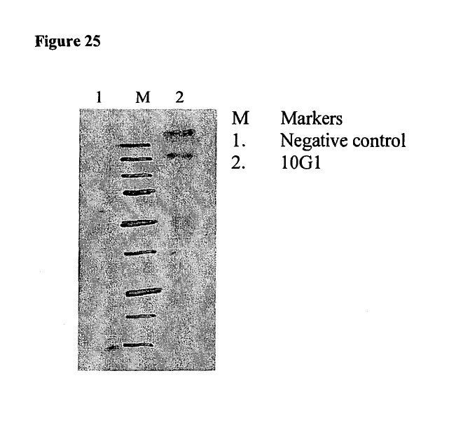

FIG. 24 is a western blot illustrating expression of 10A1 which is the GLP1 LR fusion protein GLP1-(G4S)4-GLP1R[24-145]; and FIG. 25 is a western blot illustrating expression of 10G1 GLP1/DPP4/ADA fusion protein GLP1-(G4S)5-DPP4 [39-766; S630A]-(G4S) 8-ADA[D295E; D296A)

### Materials and Methods

### Immunological Testing

Immunoassays that measure the binding of insulin to polyclonal and monoclonal antibodies are known in the art. Commercially available insulin antibodies are available to detect insulin in samples and also for use in competitive inhibition studies. For example monoclonal antibodies can be purchased at http://www.ab-direct.com/index AbD Serotec.

### Recombinant Production of Fusion Proteins

The components of the fusion proteins were generated by PCR using primers designed to anneal to the ligand or receptor and to introduce suitable restriction sites for cloning into the target vector (FIG. 14a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 14b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 14c).

Alternatively, the linker with flanking sequence, designed to anneal to the ligand or receptor domains of choice, was initially synthesised by creating an oligonucleotide duplex and this processed to generate double-stranded DNA (FIG. 15a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 15b).

### Expression and Purification of Fusion Proteins

Expression was carried out in a suitable system (e.g. mammalian CHO cells, E. coil) and this was dependant on the vector into which the insulin-fusion gene was generated. Expression was then analysed using a variety of methods which could include one or more of SDS-PAGE, Native PAGE, western blotting, ELISA well known in the art. Once a suitable level of expression was achieved the insulin fusions were expressed at a larger scale to produce enough protein for purification and subsequent analysis. Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin)._Purified protein was analysed using a variety of methods which could include one or more of Bradford's assay, SDS-PAGE, Native PAGE, western blotting, ELISA.

The fusion polypeptides include signal sequences that are processed during manufacture of the polypeptide. It will be apparent to one skilled in the art that signal sequences can be selected from a variety of sources appropriate for the particular expression system used [e.g. bacterial, mammalian]. In the not limiting examples disclosed we use the signal sequence of IL4 and growth hormone for expression in mammalian cells. For bacterial expression appropriate periplasmic signal sequences are selected.

### Characterisation of GLP-1 Fusion Proteins

Denaturing PAGE, native PAGE gels and western blotting were used to analyse the fusion polypeptides and western blotting performed with antibodies non-conformationally sensitive to the insulin fusion. Native solution state molecular weight information can be obtained from techniques such as size exclusion chromatography using a Superose G200 analytical column and analytical ultracentrifugation.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

GLP-1 LR-Fusion Expression: Western Blot of 10A1 and 10G1 from Transient Expressions in CHO Flpin Cells.

### GLP1 LR Fusion Polypeptide 10A1

50 μl of sample concentrated and then run on and SDS-PAGE gel; FIG. 24 (Lane 1). 50 μl of control media (null transfection) was also concentrated and run in parallel (Lane 2). Markers are at 250, 150, 100, 75, 50, 37, 25, 20 and 15 kDa. Immunoblot carried out with mouse anti-GLP antibody (Santa Cruz Inc.; Cat#: sc80604; dilution=1:200) and anti-mouse-HRP antibody (Abcam; dilution=1:2500). Expected Mw of 10A1 is 19 kDa.

### GLP1/DPP4/ADA Fusion Polypeptide 10G1

50 μl of sample concentrated and then run on and SDS-PAGE gel, FIG. 25 (Lane 2). 50 μl of control media (null transfection) was also concentrated and run in parallel (Lane 1). Markers are at 250, 150, 100, 75, 50, 37, 25, 20 and 15 kDa. Immunoblot carried out with mouse anti-GLP antibody (Santa Cruz Inc.; Cat#: sc80604; dilution=1:200) and anti-mouse-HRP antibody (Abcam; dilution=1:2500). Expected Mw of 10A1 is 133 kDa.

1. (canceled)

2. A fusion polypeptide comprising the amino acid sequence of a GLP-1 peptide or functional analogue thereof, linked to the amino acid sequence of a polypeptide that naturally binds GLP-1.

3. The fusion polypeptide according to claim 2, wherein the polypeptide that naturally binds GLP-1 is the GLP-1 binding domain of the GLP-1 receptor.

4. The fusion polypeptide according to claim 2, wherein the polypeptide that naturally binds the GLP-1 is an enzymatically inactive GLP-1 dipeptidyl peptidase.)

5. The fusion polypeptide according to claim 4, wherein said inactive GLP-1 dipeptidyl peptidase is modified by addition, deletion or substitution of at least one amino acid residue wherein said modification is to the active site of a GLP-1 dipeptidyl peptidase.

6. The fusion polypeptide according to claim 5, wherein said modification is to amino acid residue 630 of the amino acid sequence represented in FIG. 3a.

7. The fusion polypeptide according to claim 4, wherein said fusion polypeptide comprises or consists of the amino acid sequence represented in FIG. 3b.

8. The fusion polypeptide according to claim 4, wherein said fusion polypeptide further comprises a polypeptide that naturally binds said GLP-1 dipeptidyl peptidase wherein said polypeptide is an enzymatically inactive adenosine deaminase.

9. The fusion polypeptide according to claim 8, wherein said inactive adenosine deaminase is modified by addition, deletion or substitution of at least one amino acid residue wherein said modification is to the active site of said adenosine deaminase.

10. The fusion polypeptide according to claim 8, wherein said modification is to amino acid residues 295 and/or 296 of the amino acid sequence represented in FIG. 4a.

11. The fusion polypeptide according to claim 8, wherein said fusion polypeptide comprises of the amino acid sequence represented in FIG. 4b.

12. The fusion polypeptide according to claim 2, wherein said fusion polypeptide comprises a GLP-1 peptide comprising the amino acid sequence HAEGTFTSDVSSYLEGQAAKEFIAWLVKGR, or a modified GLP-1 peptide, wherein said modified peptide varies from said amino acid sequence by addition, deletion or substitution of at least one amino acid residue, wherein said modified GLP-1 peptide retains or has enhanced GLP-1 activity when compared to an unmodified GLP-1 peptide.

13. The fusion polypeptide according to claim 12, wherein said GLP-1 peptide comprises the amino acid sequence:
HAEGTFTSDVSSYLEGQAAKEFIAWLVKGR;
or
HAEGTFTSDVSSYLEGQAAKEFIAWLVKGRG.

14. The fusion polypeptide according to claim 12, wherein said fusion polypeptide comprises an amino acid sequence:
HGEGTFTSDLSKQMEEEAVRLFIEWLKNGGPSSGAPPPS;
or
DLSKQMEEEAVRLFIEWLKNGGPSSGAPPPS.

15. The fusion polypeptide according to claim 2, wherein GLP-1 is linked to a polypeptide that naturally binds GLP-1 by a peptide linker.

16-25. (canceled)

26. The fusion polypeptide according to claim 15, wherein said peptide linker molecule comprises of one copy of the glycosylation motif Asn-Xaa-Ser or Asn-Xaa-Thr where X is any amino acid except proline.

27. A nucleic acid molecule selected from the group consisting of:
i) a nucleic acid sequence as represented in FIG. 5b;
ii) a nucleic acid sequence as represented in FIG. 5d;
iii) a nucleic acid sequence as represented in FIG. 5f;
iv) a nucleic acid sequence as represented in FIG. 6b;
v) a nucleic acid sequence as represented in FIG. 6d;
vi) a nucleic acid sequence as represented in FIG. 6f;
vii) a nucleic acid sequence as represented in FIG. 7b;
viii) a nucleic acid sequence as represented in FIG. 7d;
ix) a nucleic acid sequence as represented in FIG. 7f;
x) a nucleic acid sequence as represented in FIG. 8b;
xi) a nucleic acid sequence as represented in FIG. 8d;
xii) a nucleic acid sequence as represented in FIG. 8f;
xiii) a nucleic acid sequence as represented in FIG. 9b;
xiv) a nucleic acid sequence as represented in FIG. 9d;
xv) a nucleic acid sequence as represented in FIG. 9f;
xvi) a nucleic acid sequence as represented in FIG. 10b;
xvii) a nucleic acid sequence as represented in FIG. 10d;
xviii) a nucleic acid sequence as represented in FIG. 10f;
xix) a nucleic acid sequence as represented in FIG. 11b;
xx) a nucleic acid sequence as represented in FIG. 11d;
xxi) a nucleic acid sequence as represented in FIG. 11f;
xxii) a nucleic acid sequence as represented in FIG. 12b;
xxiii) a nucleic acid sequence as represented in FIG. 12d;
xxiv) a nucleic acid sequence as represented in FIG. 12f; and
a nucleic acid molecule comprising a nucleic sequence that hybridizes under stringent hybridization conditions to the nucleic acid sequence represented in FIG. 5b-12f and which encodes a polypeptide that has GLP-1 receptor modulating activity.

28-29. (canceled)

30. A polypeptide encoded by a nucleic acid molecule according to claim 2.

31. A polypeptide comprising the amino acid sequence shown in the Figure selected from the group consisting of: FIG. 5a, 5c, 5e, 6a, 6c, 6e, 7a, 7c, 7e, 8a, 8c, 8e, 9a, 9c, 9e, 10a, 10c, 10e, 11a, 11c, 11e, 12a, 12c, 12e, 13a, 13c, 13e, 14a, 14c, 14e, 15a, 15c, 15e, 16a, 16c, 16e, 17a, 17c, 17e, 18a, 18c, 18e, 19a, 19c, 19e, 20a, 20c and 20e.

32. A homodimer consisting of two fusion polypeptides according to claim 2.

33-36. (canceled)

37. A pharmaceutical composition comprising a polypeptide according to claim 1, and further comprising an excipient or carrier.

38-53. (canceled)


SEQUENCE LISTING (107 sequences)

SEQ ID NO: 1 - DNA, 93 bp, Homo sapiens
catgctgaagggacctttaccagtgatgtaagttcttatttggaaggccaagctgccaaggaattcattgcttggctggtgaaaggccgagga

SEQ ID NO: 2 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 3 - protein, 87 aa, Heloderma suspectum
MKIILWLCVFGLFLATLFPISWQMPVESGLSSEDSASSESFASKIKRHGEGTFTSDLSKQMEEEAVRLFIEWLKNGGPSSGAPPPSG

SEQ ID NO: 4 - protein, 31 aa, Homo sapiens
HAEGTFTSDVSSYLEGQAAKEFIAWLVKGRG

SEQ ID NO: 5 - protein, 30 aa, Homo sapiens
HAEGTFTSDVSSYLEGQAAKEFIAWLVKGR

SEQ ID NO: 6 - protein, 39 aa, Heloderma suspectum
HGEGTFTSDLSKQMEEEAVRLFIEWLKNGGPSSGAPPPS

SEQ ID NO: 7 - protein, 31 aa, Heloderma suspectum
DLSKQMEEEAVRLFIEWLKNGGPSSGAPPPS

SEQ ID NO: 8 - protein, 463 aa, Homo sapiens [sequence omitted]

SEQ ID NO: 9 - protein, 122 aa, Homo sapiens
RPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 10 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 11 - protein, 728 aa, Homo sapiens
SRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 12 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 13 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNL

SEQ ID NO: 14 - protein, 197 aa, Artificial
  fusion protein comprising GLP-1 and GLP-1 receptor
MGLTSQLLPPLFFLLACAGNFVHGHAEGTFTSDVSSYLEGQAAKEFIAWLVKGRGGGGGSGGGGSGGGGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 15 - DNA, 594 bp, Artificial
  nucleotide sequence encoding fusion protein comprising GLP-1 and GLP-1 receptor
atgggtctcacctcccaactgcttccccctctgttcttcctgctagcatgtgccggcaactttgtccacggacatgctgaagggacctttaccagtgatgtaagttcttatttggaaggccaagctgccaaggaattcattgcttggctggtgaaaggccgaggaggtggcggaggtagtggtggcggaggtagcggtggcggaggttctggtggcggaggttcccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctactga

SEQ ID NO: 16 - protein, 205 aa, Artificial
  fusion protein comprising exendin and GLP-1 receptor
MGLTSQLLPPLFFLLACAGNFVHGHGEGTFTSDLSKQMEEEAVRLFIEWLKNGGPSSGAPPPSGGGGSGGGGSGGGGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 17 - DNA, 618 bp, Artificial
  nucleotide sequence encoding fusion protein comprising exendin and GLP-1 receptor
atgggtctcacctcccaactgcttccccctctgttcttcctgctagcatgtgccggcaactttgtccacggacatggtgaaggaacatttaccagtgacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttcccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctactga

SEQ ID NO: 18 - protein, 173 aa, Artificial
  fusion protein comprising exendin [9-39] and GLP-1 receptor
DLSKQMEEEAVRLFIEWLKNGGPSSGAPPPSGGGGSGGGGSGGGGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 19 - DNA, 594 bp, Artificial
  nucleotide sequence encoding fusion protein comprising exendin [9-39] and GLP-1 receptor
atgggtctcacctcccaactgcttccccctctgttcttcctgctagcatgtgccggcaactttgtccacggagacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttcccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctactga

SEQ ID NO: 20 - protein, 808 aa, Artificial
  fusion protein comprising GLP-1 and DPP4
MGLTSQLLPPLFFLLACAGNFVHGHAEGTFTSDVSSYLEGQAAKEFIAWLVKGRGGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 21 - DNA, 2427 bp, Artificial
  nucleotide sequence encoding fusion protein comprising GLP-1 and DPP4
atgggtctcacctcccaactgcttccccctctgttcttcctgctagcatgtgccggcaactttgtccacggacatgctgaagggacctttaccagtgatgtaagttcttatttggaaggccaagctgccaaggaattcattgcttggctggtgaaaggccgaggaggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 22 - protein, 816 aa, Artificial
  fusion protein comprising exendin and DPP4
MGLTSQLLPPLFFLLACAGNFVHGHGEGTFTSDLSKQMEEEAVRLFIEWLKNGGPSSGAPPPSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 23 - DNA, 2451 bp, Artificial
  nucleotide sequence encoding fusion protein comprising exendin and DPP4
atgggtctcacctcccaactgcttccccctctgttcttcctgctagcatgtgccggcaactttgtccacggacatggtgaaggaacatttaccagtgacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 24 - protein, 808 aa, Artificial
  fusion protein comprising exendin 9-39 and DPP4
MGLTSQLLPPLFFLLACAGNFVHGDLSKQMEEEAVRLFIEWLKNGGPSSGAPPPSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 25 - DNA, 2427 bp, Artificial
  nucleotide sequence encoding fusion  protein comprising exendin [9-39] and DPP4
atgggtctcacctcccaactgcttccccctctgttcttcctgctagcatgtgccggcaactttgtccacggagacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 26 - protein, 1211 aa, artificial
  fusion protein comprising GLP-1/DPP4 and ADA
MGLTSQLLPPLFFLLACAGNFVHGHAEGTFTSDVSSYLEGQAAKEFIAWLVKGRGGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNL

SEQ ID NO: 27 - DNA, 3636 bp, Artificial
  nucleotide sequence encoding fusion protein comprising GLP-1/DPP4 and ADA
atgggtctcacctcccaactgcttccccctctgttcttcctgctagcatgtgccggcaactttgtccacggacatgctgaagggacctttaccagtgatgtaagttcttatttggaaggccaagctgccaaggaattcattgcttggctggtgaaaggccgaggaggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 28 - protein, 1219 aa, Artificial
  fusion protein comprising exendin/DPP4 and ADA
MGLTSQLLPPLFFLLACAGNFVHGHGEGTFTSDLSKQMEEEAVRLFIEWLKNGGPSSGAPPPSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNL

SEQ ID NO: 29 - DNA, 3660 bp, Artificial
  nucleotide sequence encoding fusion protein comprising exendin/DPP4 and ADA
atgggtctcacctcccaactgcttccccctctgttcttcctgctagcatgtgccggcaactttgtccacggacatggtgaaggaacatttaccagtgacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 30 - protein, 1211 aa, Artificial
  fusion protein comprising exendin [9-39]/DPP4 and ADA
MGLTSQLLPPLFFLLACAGNFVHGDLSKQMEEEAVRLFIEWLKNGGPSSGAPPPSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNL

SEQ ID NO: 31 - DNA, 3636 bp, Artificial
  nucleotide sequence encoding fusion protein comprising exendin [9-39]/DPP4 and ADA
atgggtctcacctcccaactgcttccccctctgttcttcctgctagcatgtgccggcaactttgtccacggagacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 32 - protein, 1221 aa, Artificial
  fusion protein comprising GLP-1, ADA and DPP4
MGLTSQLLPPLFFLLACAGNFVHGHAEGTFTSDVSSYLEGQAAKEFIAWLVKGRGGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 33 - DNA, 3666 bp, Artificial
  nucleotide sequence encoding fusion protein comprising GLP-1, ADA and DPP4
atgggtctcacctcccaactgcttccccctctgttcttcctgctagcatgtgccggcaactttgtccacggacatgctgaagggacctttaccagtgatgtaagttcttatttggaaggccaagctgccaaggaattcattgcttggctggtgaaaggccgaggaggtggcggaggtagtggtggcggaggtagcggtggcggaggttctggtggcggaggttccggtggcggaggtagtggaggtggcggttctggcggtggtggatctatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 34 - protein, 1229 aa, Artificial
  fusion protein comprising exendin, ADA and DPP4
MGLTSQLLPPLFFLLACAGNFVHGHGEGTFTSDLSKQMEEEAVRLFIEWLKNGGPSSGAPPPSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 35 - DNA, 3690 bp, Artificial
  nucleotide sequence encoding fusion protein comprising exendin, ADA and DPP4
atgggtctcacctcccaactgcttccccctctgttcttcctgctagcatgtgccggcaactttgtccacggacatggtgaaggaacatttaccagtgacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtggaggtggcggttctggcggtggtggatctatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 36 - protein, 1221 aa, Artificial
  fusion protein comprising exendin 9-39, ADA and DPP4
MGLTSQLLPPLFFLLACAGNFVHGDLSKQMEEEAVRLFIEWLKNGGPSSGAPPPSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 37 - DNA, 3666 bp, Artificial
  nucleotide sequence encoding fusion protein comprising exendin 9-39, ADA and DPP4
atgggtctcacctcccaactgcttccccctctgttcttcctgctagcatgtgccggcaactttgtccacggagacttgtcaaaacagatggaagaggaggcagtgcggttatttattgagtggcttaagaacggaggaccaagtagcggggcacctccgccatcgggtggcggaggtagtggtggcggaggtagcggtggcggaggttctggtggcggaggttccggtggcggaggtagtggaggtggcggttctggcggtggtggatctatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag

SEQ ID NO: 38 - protein, 190 aa, Artificial
  fusion protein comprising GLP-1 and GLP-1 receptor
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGGGSGGGGSLVPRHAEGTFTSDVSSYLEGQAAKEFIAWLVKGRG

SEQ ID NO: 39 - DNA, 573 bp, Artificial
  nucleotide sequence encoding fusion protein comprising GLP-1 and GLP-1 receptor
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacggtggcggaggtagtggtggcggaggtagcctggtgcccagacatgctgaagggacctttaccagtgatgtaagttcttatttggaaggccaagctgccaaggaattcattgcttggctggtgaaaggccgaggatga

SEQ ID NO: 40 - protein, 198 aa, Artificial
  fusion protein comprising GLP-1 and GLP-1 receptor
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGGGSGGGGSLVPRHGEGTFTSDLSKQMEEEAVRLFIEWLKNGGPSSGAPPPS

SEQ ID NO: 41 - DNA, 597 bp, Artificial
  nucleotide sequence encoding fusion protein comprising GLP-1 and GLP-1 receptor
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacggtggcggaggtagtggtggcggaggtagcctggtgcccagacatggtgaaggaacatttaccagtgacttgtcaaaacagatggaagaggaggcagtgcggttatttattgagtggcttaagaacggaggaccaagtagcggggcacctccgccatcgtga

SEQ ID NO: 42 - protein, 190 aa, Artificial
  fusion protein comprising GLP-1 and GLP-1 receptor
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGGGSGGGGSIEPDDLSKQMEEEAVRLFIEWLKNGGPSSGAPPPS

SEQ ID NO: 43 - DNA, 573 bp, Artificial
  nucleotide sequence encoding fusion protein comprising GLP-1 and GLP-1 receptor
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacggtggcggaggtagtggtggcggaggtagcatcgagcccgacgacttgtcaaaacagatggaagaggaggcagtgcggttatttattgagtggcttaagaacggaggaccaagtagcggggcacctccgccatcgtga

SEQ ID NO: 44 - protein, 814 aa, Artificial
  fusion protein comprising DPP4 and GLP-1
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSLVPRHAEGTFTSDVSSYLEGQAAKEFIAWLVKGRG

SEQ ID NO: 45 - DNA, 2445 bp, Artificial
  nucleotide sequence encoding fusion protein comprising DPP4 and GLP-1
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtctggtgcccagacatgctgaagggacctttaccagtgatgtaagttcttatttggaaggccaagctgccaaggaattcattgcttggctggtgaaaggccgaggatag

SEQ ID NO: 46 - protein, 822 aa, Artificial
  fusion protein comprising DPP4 and exendin
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSLVPRHGEGTFTSDLSKQMEEEAVRLFIEWLKNGGPSSGAPPPS

SEQ ID NO: 47 - DNA, 2469 bp, Artificial
  nucleotide sequence encoding fusion protein comprising DPP4 and exendin
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtctggtgcccagacatggtgaaggaacatttaccagtgacttgtcaaaacagatggaagaggaggcagtgcggttatttattgagtggcttaagaacggaggaccaagtagcggggcacctccgccatcgtag

SEQ ID NO: 48 - protein, 814 aa, Artificial
  fusion protein comprising DPP4 and exendin 9-39
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSIEPDDLSKQMEEEAVRLFIEWLKNGGPSSGAPPPS

SEQ ID NO: 49 - DNA, 2445 bp, Artificial
  nucleotide sequence encoding fusion protein comprising DPP4 and exendin [9-39]
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtatcgagcccgacgacttgtcaaaacagatggaagaggaggcagtgcggttatttattgagtggcttaagaacggaggaccaagtagcggggcacctccgccatcgtag

SEQ ID NO: 50 - protein, 1227 aa, Artificial
  fusion protein comprising DPP4, ADA and GLP-1
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSLVPRHAEGTFTSDVSSYLEGQAAKEFIAWLVKGRG

SEQ ID NO: 51 - DNA, 3684 bp, Artificial
  nucleotide sequence encoding fusion protein comprising DPP4/ADA and GLP-1
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctctggtgcccagacatgctgaagggacctttaccagtgatgtaagttcttatttggaaggccaagctgccaaggaattcattgcttggctggtgaaaggccgaggatga

SEQ ID NO: 52 - protein, 1235 aa, Artificial
  fusion protein comprising DPP4, ADA and exendin
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSLVPRHGEGTFTSDLSKQMEEEAVRLFIEWLKNGGPSSGAPPPS

SEQ ID NO: 53 - DNA, 3708 bp, Artificial
  nucleotide sequence encoding fusion protein comprising DPP4, ADA and exendin
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctctggtgcccagacatggtgaaggaacatttaccagtgacttgtcaaaacagatggaagaggaggcagtgcggttatttattgagtggcttaagaacggaggaccaagtagcggggcacctccgccatcgtga

SEQ ID NO: 54 - protein, 1227 aa, Artificial
  fusion protein comprising DPP4, ADA and exendin 9-39
MATGSRTSLLLAFGLLCLPWLQEGSASRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSGGGGSGGGGSGGGGSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSIEPDDLSKQMEEEAVRLFIEWLKNGGPSSGAPPPS

SEQ ID NO: 55 - DNA, 3684 bp, Artificial
  nucleotide sequence encoding fusion protein comprising DPP4, ADA and exendin 9-39
atggctacaggctcccggacgtccctgctcctggcttttggcctgctctgcctgccctggcttcaagagggcagtgccagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtggaggtggcggttctggcggtggtggatctggaggtggcggtagtatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctatcgagcccgacgacttgtcaaaacagatggaagaggaggcagtgcggttatttattgagtggcttaagaacggaggaccaagtagcggggcacctccgccatcgtga

SEQ ID NO: 56 - protein, 1217 aa, Artificial
  fusion protein comprising ADA, DPP4 and GLP1
MATGSRTSLLLAFGLLCLPWLQEGSAMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSLVPRHAEGTFTSDVSSYLEGQAAKEFIAWLVKGRG

SEQ ID NO: 57 - DNA, 3654 bp, Artificial
  nucleotide sequence encoding fusion protein comprising ADA, DPP4 and GLP1
atggctacaggctcccggacgtccctgctcctggcttttggcctgctctgcctgccctggcttcaagagggcagtgccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtctggtgcccagacatgctgaagggacctttaccagtgatgtaagttcttatttggaaggccaagctgccaaggaattcattgcttggctggtgaaaggccgaggatag

SEQ ID NO: 58 - protein, 1225 aa, Artificial
  fusion protein comprising ADA, DPP4 and exendin
MATGSRTSLLLAFGLLCLPWLQEGSAMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSLVPRHGEGTFTSDLSKQMEEEAVRLFIEWLKNGGPSSGAPPPS

SEQ ID NO: 59 - DNA, 3678 bp, Artificial
  nucleotide sequence encoding fusion protein comprising ADA, DPP4 and exendin
atggctacaggctcccggacgtccctgctcctggcttttggcctgctctgcctgccctggcttcaagagggcagtgccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtctggtgcccagacatggtgaaggaacatttaccagtgacttgtcaaaacagatggaagaggaggcagtgcggttatttattgagtggcttaagaacggaggaccaagtagcggggcacctccgccatcgtag

SEQ ID NO: 60 - protein, 1217 aa, Artificial
  fusion protein comprising ADA, DPP4 and exendin 9-39
MATGSRTSLLLAFGLLCLPWLQEGSAMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTEAPLIFKSTLDTDYQMTKRDMGFTEEEFKRLNINAAKSSFLPEDEKRELLDLLYKAYGMPPSASAGQNLGGGGSGGGGSGGGGSGGGGSGGGGSGGGGSGGGGSGGGGSSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWAYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPGGGGSGGGGSGGGGSGGGGSGGGGSIEPDDLSKQMEEEAVRLFIEWLKNGGPSSGAPPPS

SEQ ID NO: 61 - DNA, 3654 bp, Artificial
  nucleotide sequence encoding fusion protein comprising ADA/DPP4 and exendin 9-39
atggctacaggctcccggacgtccctgctcctggcttttggcctgctctgcctgccctggcttcaagagggcagtgccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtggccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagaagccccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggtggcggaggtagtggtggcggaggtagcggtggcggaggttctggtggcggaggttccggtggcggaggtagtggaggtggcggttctggcggtggtggatctggaggtggcggtagtagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctgggcctatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttacctggtggcggaggtagtggtggcggaggtagcggtggcggaggttctggtggcggaggttccggtggcggaggtagtatcgagcccgacgacttgtcaaaacagatggaagaggaggcagtgcggttatttattgagtggcttaagaacggaggaccaagtagcggggcacctccgccatcgtag

SEQ ID NO: 62 - protein, 197 aa, Artificial
  fusion protein comprising GLP1 and GLP1 receptor
MGLTSQLLPPLFFLLACAGNFVHGHAEGTFTSDVSSYLEGQAAKEFIAWLVKGRGGGGGSGGGGSGGNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 63 - protein, 205 aa, Artificial
  fusion protein comprising exendin and GLP1 receptor
MGLTSQLLPPLFFLLACAGNFVHGHGEGTFTSDLSKQMEEEAVRLFIEWLKNGGPSSGAPPPSGGGGSGGGGSGGNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 64 - protein, 197 aa, Artificial
  fusion protein comprising exendin 9-39 and GLP1 receptor
MGLTSQLLPPLFFLLACAGNFVHGDLSKQMEEEAVRLFIEWLKNGGPSSGAPPPSGGGGSGGGGSGGNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 65 - protein, 190 aa, Artificial
  fusion protein comprising GLP1 and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGSGGGGSLVPRHAEGTFTSDVSSYLEGQAAKEFIAWLVKGRG

SEQ ID NO: 66 - protein, 198 aa, Artificial
  fusion protein comprising exendin and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGSGGGGSLVPRHGEGTFTSDLSKQMEEEAVRLFIEWLKNGGPSSGAPPPS

SEQ ID NO: 67 - protein, 190 aa, Artificial
  fusion protein comprising GLP1 receptor and exendin 9-39 gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGSGGGGSIEPDDLSKQMEEEAVRLFIEWLKNGGPSSGAPPPS

SEQ ID NO: 68 - protein, 197 aa, Artificial
  fusion protein comprising GLP1 and GLP1 receptor gly linked
MGLTSQLLPPLFFLLACAGNFVHGHAEGTFTSDVSSYLEGQAAKEFIAWLVKGRGGGGGSGGGGSGGNGTGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 69 - protein, 205 aa, Artificial
  fusion protein comprising exendin and GLP-1 receptor gly linked
MGLTSQLLPPLFFLLACAGNFVHGHGEGTFTSDLSKQMEEEAVRLFIEWLKNGGPSSGAPPPSGGGGSGGGGSGGNGTGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 70 - protein, 197 aa, Artificial [sequence omitted]

SEQ ID NO: 71 - protein, 190 aa, Artificial
  fusion protein comprising GLP 1 receptor and GLP1 gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGTGGGGSLVPRHAEGTFTSDVSSYLEGQAAKEFIAWLVKGRG

SEQ ID NO: 72 - protein, 198 aa, Artificial
  fusion protein comprising GLP1 and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGTGGGGSLVPRHGEGTFTSDLSKQMEEEAVRLFIEWLKNGGPSSGAPPPS

SEQ ID NO: 73 - protein, 190 aa, Artificial
  fusion protein comprising extendin9-39  and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNGTGGGGSIEPDDLSKQMEEEAVRLFIEWLKNGGPSSGAPPPS

SEQ ID NO: 74 - protein, 197 aa, Artificial
  fusion protein comprising GLP1 and GLP1 receptor gly linked
MGLTSQLLPPLFFLLACAGNFVHGHAEGTFTSDVSSYLEGQAAKEFIAWLVKGRGGGGGSGGGGSGWNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 75 - protein, 205 aa, Artificial
  fusion protein comprising exendin and GLP1 receptor gly linked
MGLTSQLLPPLFFLLACAGNFVHGHGEGTFTSDLSKQMEEEAVRLFIEWLKNGGPSSGAPPPSGGGGSGGGGSGWNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 76 - protein, 197 aa, Artificial
  fusion protein comprising exendin 9-39 and GLP-1 receptor gly linked
MGLTSQLLPPLFFLLACAGNFVHGDLSKQMEEEAVRLFIEWLKNGGPSSGAPPPSGGGGSGGGGSGWNGSGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 77 - protein, 190 aa, Artificial
  fusion protein comprising GLP1 receptor and GLP1 gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGWNGSGGGGSLVPRHAEGTFTSDVSSYLEGQAAKEFIAWLVKGRG

SEQ ID NO: 78 - protein, 198 aa, Artificial
  fusion protein comprising exendin and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGWNGSGGGGSLVPRHGEGTFTSDLSKQMEEEAVRLFIEWLKNGGPSSGAPPPS

SEQ ID NO: 79 - protein, 190 aa, Artificial
  fusion protein comprising exendin 9-39 and GLP1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGWNGSGGGGSIEPDDLSKQMEEEAVRLFIEWLKNGGPSSGAPPPS

SEQ ID NO: 80 - protein, 197 aa, Artificial
  fusion protein comprising GLP1 and GLP1 receptor gly linked
MGLTSQLLPPLFFLLACAGNFVHGHAEGTFTSDVSSYLEGQAAKEFIAWLVKGRGGGGGSGGGGSGGNATGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 81 - protein, 205 aa, Artificial
  fusion protein comprising exendin and GLP1 receptor
MGLTSQLLPPLFFLLACAGNFVHGHGEGTFTSDLSKQMEEEAVRLFIEWLKNGGPSSGAPPPSGGGGSGGGGSGGNATGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 82 - protein, 197 aa, Artificial
  fusion protein comprising exendin and GLP1 receptor
MGLTSQLLPPLFFLLACAGNFVHGDLSKQMEEEAVRLFIEWLKNGGPSSGAPPPSGGGGSGGGGSGGNATGGGGSRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLY

SEQ ID NO: 83 - protein, 190 aa, Artificial
  fusion protein comprising GLP 1 receptor and GLP 1 gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNATGGGGSLVPRHAEGTFTSDVSSYLEGQAAKEFIAWLVKGRG

SEQ ID NO: 84 - protein, 198 aa, Artificial [sequence omitted]

SEQ ID NO: 85 - protein, 190 aa, Artificial
  fusion protein comprising exendin 9-39 and GLP 1 receptor gly linked
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYGGNATGGGGSIEPDDLSKQMEEEAVRLFIEWLKNGGPSSGAPPPS

SEQ ID NO: 86 - protein, 24 aa, Homo sapiens
MGLTSQLLPPLFFLLACAGNFVHG

SEQ ID NO: 87 - DNA, 72 bp, Homo sapiens
atgggtctcacctcccaactgcttccccctctgttcttcctgctagcatgtgccggcaactttgtccacgga

SEQ ID NO: 88 - protein, 5 aa, Artificial
  gly rich peptide linker
GGGGS

SEQ ID NO: 89 - protein, 5 aa, Artificial
  glycosylation motif
XXXXX

SEQ ID NO: 90 - protein, 5 aa, Artificial
  glycosylation motif
NXSXX

SEQ ID NO: 91 - protein, 5 aa, Artificial
  glycosylation motif
XNXSX

SEQ ID NO: 92 - protein, 5 aa, Artificial
  glycosylation motif
XXNXS

SEQ ID NO: 93 - protein, 5 aa, Artificial
  glycosylation motif
NXTXX

SEQ ID NO: 94 - protein, 5 aa, Artificial
  glycosylation motif
XNXTX

SEQ ID NO: 95 - protein, 5 aa, Artificial
  glycosylation motif
XXNXT

SEQ ID NO: 96 - protein, 5 aa, Artificial
  glycosylation motif
NXSGS

SEQ ID NO: 97 - protein, 5 aa, Artificial
  glycosylation motif
GNXSS

SEQ ID NO: 98 - protein, 5 aa, Artificial
  glycosylation motif
GGNXS

SEQ ID NO: 99 - protein, 5 aa, Artificial
  glycosylation motif
NXTGS

SEQ ID NO: 100 - protein, 5 aa, Artificial
  glycosylation motif
GGNXT

SEQ ID NO: 101 - protein, 5 aa, Artificial
  glycosylation motif
GNXTS

SEQ ID NO: 102 - protein, 5 aa, Artificial
  glycosylation motif
NXSSG

SEQ ID NO: 103 - protein, 5 aa, Artificial
  glycosylation motif
SNXSG

SEQ ID NO: 104 - protein, 5 aa, Artificial
  glycosylation motif
SSNXS

SEQ ID NO: 105 - protein, 5 aa, Artificial
  glycosylation motif
NXTSG

SEQ ID NO: 106 - protein, 5 aa, Artificial
  glycosylation motif
SNXTG

SEQ ID NO: 107 - protein, 5 aa, Artificial
  glycosylation motif
SSNXT